SEQUENCE LISTING (38 sequences)

SEQ ID NO: 1 - DNA, 4939 bp, Artificial Sequence
  MXPyV Sequence
gaggcttaggcctccggcccccggcttatatagaaaaaattttagcttattgttttgctacttaacctcaggtaggtcaacagctattgttggcaagctattgttggcaagtattggtattaatcacccagacaactcagaagtttccacctcttggaggcggtccagagttaacctgtgactgttggcgggaagccaataacagcaactttgacatttcatcacgagccctttaaacgccctctaggcggagacggaagacaattgactcttggcacggacggaagaggaatgccgtctgctcaccttagttaacacgagattttctttggaaatactccaaagtacagtaagtatatgggtgctgtaatctctgttattcttgatttaatagaattaatatcagttacaggctttgaagcagaaactataatttcaggggaggcagctgctatagtagaatcacagctttcatctttggcagttatagaaaatgcttcggcagccgaagttttatctacttttggattaaatgaaacctcttattctttattagtaaattttcctcaggcctttgaaaatgctgtatatactgctcaattaattcaaactatttctggggctagttctctgattgctgctggtatagaaacgcaaccttttcaagtatttgatgctggtagtaatatggctttacagcaatggagaccagactattttgatttatttattcctggatatagacactttgaatattattttaatgttctttctggttggggagaaagtctagtaaatacagtttctagagctttttgggaggctttattatcagaaactagaaacactgcaagacttttagcaagcagtgctgtagataatgtttataatgttggtgaacagggactccagaatattcaaaatgctctggtgggattaatagagagtgcaagatgggctttaagatttccggggaatgtatatcataatttagaaatgtactatacacaattaccaggccttactcctccacaagtaaggtcaattcaaaggcgcttagaagaagcgagaaattatggaccttctgttcaagtggataattctgaaaactcagttttttcagaacatttaagtggagactatatatttagaggagaagctccaggaggagcaagacaaagacagactcctgattggatgctacaattaattttaggaattcttggagatataactcctttttttaaagaagttattgaagaagtagaaggagaggaaaatgccgcctaaaagaaagactgtttgtactaaaactgtttgtaccagagcagaagcccccaacaaaaaagtctgtgaaaaacccacgtgttccagatcttgcagaatgtcatgtaacaaatgtccttgtataccgtgtccagttcctactaaagttcctagaattgtttctaagggtgggatagaagttttaaatataattccagggccggacaccacaatgacagttgaggtcattcttcaaccaaggatgggcaatgatgtaaaaacaaacaaatggtatggctacagtgatccaataacagtaacaaatactcctggtattaatcagctgcccacttatagtacagctaaaattaaactgccacccttaaatgaagatatgacctgtgaaaatttgtatatgtgggaggcagttgttcttaaaactgaattaataggagttactagcttaataactttgcatactccaggtgttgcagatcctgctactgcgccagctttaccagtggaaggggtttcatttcatttttttgcagtgggaggacagcctttagatttacaatattgtgttcctgcagctgatattgtttacccagatggaacaggcagttttgcaagttcagggggaactcagcaaacctatgatgcctcatttaaagccattttagatagagatggatactatcctgtggaggcctgggctccagatccagtaaaaaatgataattctagatactatggaactgttactggaggtactacaacacctcctgtgcttagtacaacaaatgctgtttctacagtattactggatgatagaggtgtgggacccctgtgtaagggagatgggctatatgtaacagcagtagatatttgtggagtgtttcaaatgcctgataatactaggagacacaggggcctagcaagatactttcaggtacagcttagacaaagagctgttagaaatccttatcctgtaaatagtttgttaaacagtttactgacaaaacaaatacctagcattgatggacagccaatggatgggactgataatcaagtacaggatgtaactgtgttccaaggaacagaacctcttccaggagaccccactttaactagacatatggatttgcgttgctgcccgggaaccccagtaacagatatgccaagtgacgacactccaacaccagttgctccagcagcaagataatttattgtgaattaattccagagtcttgttgtgtatcctcgtcagcatctacacatattaaaatatctttcaatggatcttccccagattctatgtttcttcttatatcatgatacattccaaagggtacatatttttcaattgtttctttccaatatcttacattatcatgaatttcaggatgaaatgcaattacaggttgccaccaacaaagaagcaataataatgtaacaccactttgaacaattctttttctgagcaactcagaatttttttccaaagaatttcttaagaacaacttaggcctgaaatttatagtttttatcatcctagcttgcaaagttggaggaataaaatagtcattcatagtaataatacctgggggaaatatttggctctttttattcacatgttttttttcaagatttacttttacacaaccatccaaatgatcccttaaattatctaagttggacatacccattcctggagttaaagatgagttagagccctcgttttgaccttttacatcttcaaaaacaaccatatattcatcaattgcacaaccaatttcaaaagctaacttatctggaggacagttaacatttagagtttttcccccaagcaaatcaagtatagcagcagctacagttgttttaccactattaattgggcctttaaataaaacatatctcctcttaggtacattttcagtcatggcctttattataaataaaataatttcatcaaaatgaggcatcaataatgataaccaagctactccagccatgtattgacaaatttcaacctcaccacaaatgtcttccattttttcaaacatatgtttaaaccttaataccaacagatgttctctggtactttctagtattaataatcttctctgagcagtgacccagtctgtagcttgctgacaaattgttttttgatttttacaatccttaaaaagttttgaattaatattttgagtttcatggaatttataatggtgttttaattttttttgttcacattttgagcacccctctacatcttttgcaaagtctaataacattcccataagtagcaagggatcctcacattgaacttctactgcaaattcacaaatttgctgccaattcacaactttatctttttcttcctcacagaaatctgttttacttaaaccatgttcctgactctgtttaattaatttaaaaggagttttacaaagagcatagtaacattcataggcctttaagcaaccttttactaatagaaagctcactgtacacagatttgaacaataattttttatagcacttactctatgttttccacttaataataaaaataataaactgtcaccatcaaattcatgcaaactataaaacatagctttaaattttaaaggcactttttcatacaagaattgccccttttcccttgtagtatataaaacaaaagaattcagagttttattactaaaaatagcattactaagaaattcaaggagtacttcaggaaaatctacaggattaaagtttcttggcctttttggaggagtacaggtagaattgctgctggattctctgggtctcttcttaggagtgttggtatcatttgcagtctgagaagcactttgagaaggccccggttcttcctcatcactgggagcaaaagattcattacaacttaaatcttcatcccatcctctattaaattcttcccaccattgatcccattcaggagtcccataagtgggatttccctaaaataaaagagaaagaacttacccacaaaattaattctcccagcaagcgaagcagatcaaattcagtattgtgcattatatgcttccaccaaaagaatgaagtatagccaaattcttgtccaaaccaaagcaaaaagcatttgtagcaaaaacattcaccccaaacaagacattgcttttgtttcgctagtttatcatttctgtgctgctttttaagcaagcagcaaacacagccacatttgtgtcttaataaatcacttgcacacaaaggccaaatgtatataattttttcctcaaagctaggtcctagcacatctcctaaagttactacatcgtctataaagtaaccaacctttgcaggaaaataaacttctccttctcttctaagcttttcaattgtagtatacattttagaaaacggctcattcaagcgtttcattttttctccatctccccctttgtcagggtgcagttttaaacaagtctgcctgtatttatattgcattaagggaatatttccccaagcagcagtatttaagcttaaaagagccataagctcttttacttcatctctagaaagtactctatccatccttgctgaatttgcaagtagtaaaaagtttgcagacgcggtaaagatggctcccagagtccttcctcttttcaccggaaagaca

SEQ ID NO: 2 - DNA, 933 bp, Artificial Sequence
  MXPyV Sequence
atgggtgctgtaatctctgttattcttgatttaatagaattaatatcagttacaggctttgaagcagaaactataatttcaggggaggcagctgctatagtagaatcacagctttcatctttggcagttatagaaaatgcttcggcagccgaagttttatctacttttggattaaatgaaacctcttattctttattagtaaattttcctcaggcctttgaaaatgctgtatatactgctcaattaattcaaactatttctggggctagttctctgattgctgctggtatagaaacgcaaccttttcaagtatttgatgctggtagtaatatggctttacagcaatggagaccagactattttgatttatttattcctggatatagacactttgaatattattttaatgttctttctggttggggagaaagtctagtaaatacagtttctagagctttttgggaggctttattatcagaaactagaaacactgcaagacttttagcaagcagtgctgtagataatgtttataatgttggtgaacagggactccagaatattcaaaatgctctggtgggattaatagagagtgcaagatgggctttaagatttccggggaatgtatatcataatttagaaatgtactatacacaattaccaggccttactcctccacaagtaaggtcaattcaaaggcgcttagaagaagcgagaaattatggaccttctgttcaagtggataattctgaaaactcagttttttcagaacatttaagtggagactatatatttagaggagaagctccaggaggagcaagacaaagacagactcctgattggatgctacaattaattttaggaattcttggagatataactcctttttttaaagaagttattgaagaagtagaaggagaggaaaatgccgcctaa

SEQ ID NO: 3 - DNA, 603 bp, Artificial Sequence
  MXPyV Sequence
atggctttacagcaatggagaccagactattttgatttatttattcctggatatagacactttgaatattattttaatgttctttctggttggggagaaagtctagtaaatacagtttctagagctttttgggaggctttattatcagaaactagaaacactgcaagacttttagcaagcagtgctgtagataatgtttataatgttggtgaacagggactccagaatattcaaaatgctctggtgggattaatagagagtgcaagatgggctttaagatttccggggaatgtatatcataatttagaaatgtactatacacaattaccaggccttactcctccacaagtaaggtcaattcaaaggcgcttagaagaagcgagaaattatggaccttctgttcaagtggataattctgaaaactcagttttttcagaacatttaagtggagactatatatttagaggagaagctccaggaggagcaagacaaagacagactcctgattggatgctacaattaattttaggaattcttggagatataactcctttttttaaagaagttattgaagaagtagaaggagaggaaaatgccgcctaa

SEQ ID NO: 4 - DNA, 1212 bp, Artificial Sequence
  MXPyV Sequence
atgccgcctaaaagaaagactgtttgtactaaaactgtttgtaccagagcagaagcccccaacaaaaaagtctgtgaaaaacccacgtgttccagatcttgcagaatgtcatgtaacaaatgtccttgtataccgtgtccagttcctactaaagttcctagaattgtttctaagggtgggatagaagttttaaatataattccagggccggacaccacaatgacagttgaggtcattcttcaaccaaggatgggcaatgatgtaaaaacaaacaaatggtatggctacagtgatccaataacagtaacaaatactcctggtattaatcagctgcccacttatagtacagctaaaattaaactgccacccttaaatgaagatatgacctgtgaaaatttgtatatgtgggaggcagttgttcttaaaactgaattaataggagttactagcttaataactttgcatactccaggtgttgcagatcctgctactgcgccagctttaccagtggaaggggtttcatttcatttttttgcagtgggaggacagcctttagatttacaatattgtgttcctgcagctgatattgtttacccagatggaacaggcagttttgcaagttcagggggaactcagcaaacctatgatgcctcatttaaagccattttagatagagatggatactatcctgtggaggcctgggctccagatccagtaaaaaatgataattctagatactatggaactgttactggaggtactacaacacctcctgtgcttagtacaacaaatgctgtttctacagtattactggatgatagaggtgtgggacccctgtgtaagggagatgggctatatgtaacagcagtagatatttgtggagtgtttcaaatgcctgataatactaggagacacaggggcctagcaagatactttcaggtacagcttagacaaagagctgttagaaatccttatcctgtaaatagtttgttaaacagtttactgacaaaacaaatacctagcattgatggacagccaatggatgggactgataatcaagtacaggatgtaactgtgttccaaggaacagaacctcttccaggagaccccactttaactagacatatggatttgcgttgctgcccgggaaccccagtaacagatatgccaagtgacgacactccaacaccagttgctccagcagcaagataa

SEQ ID NO: 5 - DNA, 2007 bp, Artificial Sequence
  MXPyV Sequence
ggaaatcccacttatgggactcctgaatgggatcaatggtgggaagaatttaatagaggatgggatgaagatttaagttgtaatgaatcttttgctcccagtgatgaggaagaaccggggccttctcaaagtgcttctcagactgcaaatgataccaacactcctaagaagagacccagagaatccagcagcaattctacctgtactcctccaaaaaggccaagaaactttaatcctgtagattttcctgaagtactccttgaatttcttagtaatgctatttttagtaataaaactctgaattcttttgttttatatactacaagggaaaaggggcaattcttgtatgaaaaagtgcctttaaaatttaaagctatgttttatagtttgcatgaatttgatggtgacagtttattatttttattattaagtggaaaacatagagtaagtgctataaaaaattattgttcaaatctgtgtacagtgagctttctattagtaaaaggttgcttaaaggcctatgaatgttactatgctctttgtaaaactccttttaaattaattaaacagagtcaggaacatggtttaagtaaaacagatttctgtgaggaagaaaaagataaagttgtgaattggcagcaaatttgtgaatttgcagtagaagttcaatgtgaggatcccttgctacttatgggaatgttattagactttgcaaaagatgtagaggggtgctcaaaatgtgaacaaaaaaaattaaaacaccattataaattccatgaaactcaaaatattaattcaaaactttttaaggattgtaaaaatcaaaaaacaatttgtcagcaagctacagactgggtcactgctcagagaagattattaatactagaaagtaccagagaacatctgttggtattaaggtttaaacatatgtttgaaaaaatggaagacatttgtggtgaggttgaaatttgtcaatacatggctggagtagcttggttatcattattgatgcctcattttgatgaaattattttatttataataaaggccatgactgaaaatgtacctaagaggagatatgttttatttaaaggcccaattaatagtggtaaaacaactgtagctgctgctatacttgatttgcttgggggaaaaactctaaatgttaactgtcctccagataagttagcttttgaaattggttgtgcaattgatgaatatatggttgtttttgaagatgtaaaaggtcaaaacgagggctctaactcatctttaactccaggaatgggtatgtccaacttagataatttaagggatcatttggatggttgtgtaaaagtaaatcttgaaaaaaaacatgtgaataaaaagagccaaatatttcccccaggtattattactatgaatgactattttattcctccaactttgcaagctaggatgataaaaactataaatttcaggcctaagttgttcttaagaaattctttggaaaaaaattctgagttgctcagaaaaagaattgttcaaagtggtgttacattattattgcttctttgttggtggcaacctgtaattgcatttcatcctgaaattcatgataatgtaagatattggaaagaaacaattgaaaaatatgtaccctttggaatgtatcatgatataagaagaaacatagaatctggggaagatccattgaaagatattttaatatgtgtagatgctgacgaggatacacaacaagactctggaattaattcacaataaatggatagagtactttctagagatgaagtaaaagagcttatggctcttttaagcttaaatactgctgcttggggaaatattcccttaatgcaatataaatacaggcagacttgtttaaaactgcaccctgacaaagggggagatggagaaaaaatgaaacgcttgaatgagccgttttctaaaatgtatactacaattgaaaagcttagaagagaaggagaagtttattttcctgcaaag

SEQ ID NO: 6 - DNA, 621 bp, Artificial Sequence
  MXPyV Sequence
atggatagagtactttctagagatgaagtaaaagagcttatggctcttttaagcttaaatactgctgcttggggaaatattcccttaatgcaatataaatacaggcagacttgtttaaaactgcaccctgacaaagggggagatggagaaaaaatgaaacgcttgaatgagccgttttctaaaatgtatactacaattgaaaagcttagaagagaaggagaagtttattttcctgcaaaggttggttactttatagacgatgtagtaactttaggagatgtgctaggacctagctttgaggaaaaaattatatacatttggcctttgtgtgcaagtgatttattaagacacaaatgtggctgtgtttgctgcttgcttaaaaagcagcacagaaatgataaactagcgaaacaaaagcaatgtcttgtttggggtgaatgtttttgctacaaatgctttttgctttggtttggacaagaatttggctatacttcattcttttggtggaagcatataatgcacaatactgaatttgatctgcttcgcttgctgggagaattaattttgtgggtaagttctttctcttttattttagggaaatcccacttatgggactcctga

SEQ ID NO: 7 - DNA, 240 bp, Artificial Sequence
  MXPyV Sequence
atggatagagtactttctagagatgaagtaaaagagcttatggctcttttaagcttaaatactgctgcttggggaaatattcccttaatgcaatataaatacaggcagacttgtttaaaactgcaccctgacaaagggggagatggagaaaaaatgaaacgcttgaatgagccgttttctaaaatgtatactacaattgaaaagcttagaagagaaggagaagtttattttcctgcaaag

SEQ ID NO: 8 - DNA, 24 bp, Artificial Sequence
  MX-VP1-77F Primer
ggttgaagaatgacctcaactgtc

SEQ ID NO: 9 - DNA, 23 bp, Artificial Sequence
  MX-VP1-231R Primer
gtatatgtgggaggcagttgttc

SEQ ID NO: 10 - DNA, 20 bp, Artificial Sequence
  MX-VP3-F Primer
gacagactcctgattggatg

SEQ ID NO: 11 - DNA, 24 bp, Artificial Sequence
  MX-largeT-F Primer
cctgtagattttcctgaagtactc

SEQ ID NO: 12 - DNA, 22 bp, Artificial Sequence
  MX-VP1-1F Primer
cggacaccacaatgacagttga

SEQ ID NO: 13 - DNA, 22 bp, Artificial Sequence
  MX-VP3-R Primer
gcttctgctctggtacaaacag

SEQ ID NO: 14 - DNA, 23 bp, Artificial Sequence
  MX-largeT-R1 Primer
gctcactgtacacagatttgaac

SEQ ID NO: 15 - DNA, 19 bp, Artificial Sequence
  MX-2500-1F Primer
aagctacagactgggtcac

SEQ ID NO: 16 - DNA, 18 bp, Artificial Sequence
  MX-2500-2R Primer
tcttctctgagcagtgac

SEQ ID NO: 17 - DNA, 18 bp, Artificial Sequence
  MX-2500-1R Primer
ctacagtattactggatg

SEQ ID NO: 18 - DNA, 18 bp, Artificial Sequence
  MX-2500-2F Primer
ctgctgttacatatagcc

SEQ ID NO: 19 - DNA, 18 bp, Artificial Sequence
  MX-2000-2R Primer
gtccagagttaacctgtg

SEQ ID NO: 20 - DNA, 19 bp, Artificial Sequence
  MX-2000-3R Primer
cctggatatagacactttg

SEQ ID NO: 21 - DNA, 18 bp, Artificial Sequence
  MX-2500-4004 Primer
gtgtcgtcacttggcata

SEQ ID NO: 22 - DNA, 18 bp, Artificial Sequence
  MX-2500-3607 Primer
atagtaataatacctggg

SEQ ID NO: 23 - DNA, 18 bp, Artificial Sequence
  MX-smallT-1724 Primer
ttaaaactgcaccctgac

SEQ ID NO: 24 - DNA, 19 bp, Artificial Sequence
  MX-Scr-VP1-523F Primer
gaggcctgggctccagatc

SEQ ID NO: 25 - DNA, 20 bp, Artificial Sequence
  MX-Scr-VP1-660R Primer
cccacacctctatcatccag

SEQ ID NO: 26 - DNA, 24 bp, Artificial Sequence
  MX-Conf-largeT-1F Primer
cctgtagattttcctgaagtactc

SEQ ID NO: 27 - DNA, 23 bp, Artificial Sequence
  MX-Conf-LargeT-2R Primer
gctcactgtacacagatttgaac

SEQ ID NO: 28 - DNA, 21 bp, Artificial Sequence
  MX-Conf-VP1-661Fdeg Primer
cccytgtgtaarggagatggg

SEQ ID NO: 29 - DNA, 25 bp, Artificial Sequence
  MX-Conf-VP1-807Rdeg Primer
aggrtaaggatttytaacagcyctt

SEQ ID NO: 30 - DNA, 23 bp, Artificial Sequence
  Calici-F Primer
gattactccargtgggaytcmac

SEQ ID NO: 31 - DNA, 22 bp, Artificial Sequence
  Calici-R Primer
tgacratktmatcatcmccrta

SEQ ID NO: 32 - DNA, 25 bp, Artificial Sequence
  Adeno-F Primer
gccgcagtggtcttacatgcacatc

SEQ ID NO: 33 - DNA, 23 bp, Artificial Sequence
  Adeno-R Primer
cagcacgccgcggatgtcaaagt

SEQ ID NO: 34 - DNA, 20 bp, Artificial Sequence
  Entero- F Primer
cggcccctgaatgcggctaa

SEQ ID NO: 35 - DNA, 19 bp, Artificial Sequence
  Entero-R Primer
attgtcaccataagcagcc

SEQ ID NO: 36 - DNA, 22 bp, Artificial Sequence
  Rota -F Primer
aagtagctggatttgattattc

SEQ ID NO: 37 - DNA, 26 bp, Artificial Sequence
  Random Primer
gttcccactggaggatannnnnnnnn

SEQ ID NO: 38 - DNA, 14 bp, Artificial Sequence
  Pentanucleotide Palindrome Polyomavirus
gaggcnnnngcctc

(19) United States
(12) Patent Application Publication
(10) Pub. No.: US 2014/0065599 A1
(43) Pub. Date: Mar. 6, 2014

(54) NOVEL POLYMAVIRUS ASSOCIATED WITH DIARRHEA IN CHILDREN

(71) Applicant: THE REGENTS OF THE UNIVERSITY OF CALIFORNIA, Oakland, CA (US)

(72) Inventors: Charles CHIU, San Francisco, CA (US); Guixia Yu, Hercules, CA (US); Alexander Greninger, San Francisco, CA (US); Pavel Isa, Cuernavaca (MX); Carlos F. Arias, Cuernavaca (MX); Joseph De Risi, San Francisco, CA (US); Juliet Parsonnet, Stanford, CA (US); Steve Miller, San Mateo, CA (US)

(21) Appl. No.: 13/973,544

(22) Filed: Aug. 22, 2013

Related U.S. Application Data
(60) Provisional application No. 61/692,170, filed on Aug. 22, 2012.

(51) Int. Cl. G01N 33/569; C07K 16/08; C07K 2/00

(52) U.S. Cl. CPC G01N 33/56983; C07K 2/00; C07K 16/081
USPC 435/5; 536/23.72; 435/320.1; 530/300; 530/389.4; 530/388.3; 536/24.33; 435/252.3; 435/325; 435/254.2; 435/348

(57) ABSTRACT

Provided is a new polyomavirus, provisionally named MX polyomavirus, (MXPyV). Further provided are cDNA nucleic acid sequences, recombinant proteins, expression vectors and host cells, recombinant anti-MXPyV antibodies, vaccines, compositions, methods of detecting MXPyV, methods for assaying for anti-MXPyV compounds, and methods for treating or preventing a MXPyV infection.

### CROSS REFERENCE TO RELATED APPLICATIONS

This application claims benefit under 35 USC 119(e) to U.S. Provisional Application No. 61/692,170 filed Aug. 22, 2012, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under grant nos. R01 AI042801, R01 HL 105770, R56AI089532, and R01 HL105704 awarded by National Institutes of Health. The government has certain rights in the invention.

### FIELD OF THE INVENTION

The present invention relates to the discovery of a new polyomavirus, provisionally named MX polyavirus (MXPyV), nucleic acids, proteins, vaccines, compositions, kits, methods of detecting and diagnosing MXPyV infection, methods of treating or preventing MXPyV infection, and methods for identifying anti-MXPyV compounds.

### BACKGROUND OF THE INVENTION

Polyomaviruses are small, circular DNA viruses that can cause persistent infections in both animals and humans, and are also potentially oncogenic (21). In humans, polyomaviruses are associated with a broad spectrum of diseases ranging from progressive multifocal leukoencephalopathy (PML) (JCV, JC virus) to nephropathy (BKV, BK virus), to Merkel cell cancer (MCV, Merkel cell virus) (9, 23, 31, 32). Ongoing efforts to identify and characterize novel polyomaviruses are important as they may yield valuable insights into the establishment of latent infections and viral carcinogenesis.

The human polyomaviruses JCV and BKV, initially described in 1971 (25, 41), are closely related to each other genetically and show 70-80% seroprevalence in adults (37). BKV can establish a chronic infection in the kidneys (47), and causes nephropathy and hemorrhagic cystitis in transplant patients (9), although it can also be detected in urine from healthy individuals (37). JCV also latently infect the kidneys (44), but in immunocompromised individuals, especially in patients with HIV, can invade the central nervous system and cause PML, a life-threatening demyelinating illness associated with headaches, memory loss, and neurological deficits (31). Up until 2007, the only two polyomaviruses known to infect humans were JCV and BKV, but recent advances in sequencing technologies have since led to the discovery of seven additional human polyomaviruses. The WU and KI polyomaviruses were initially described in 2007 in children with acute respiratory illness (4, 26), but the exact pathogenic role of these viruses in respiratory disease remains controversial (6). These viruses have been found to infect the respiratory tract of up to 7% of children (1, 4, 8, 26, 30, 45, 58, 59), with or without respiratory symptoms, and, like BCV and JCV, seroprevalence rates in both children and adult populations are high, exceeding 50% (36). MCV was first described in 2008 in association with a rare but aggressive type of skin cancer called Merkel cell carcinoma (MCC) (23). In tumor cells, MCV integrates into the host genome and is unable to replicate due to truncation mutations in the viral T antigen (51). The direct etiologic role of MCV in oncogenesis was demonstrated by cell death and regression of MCC tumors upon knockdown of the viral T antigen (32). Since the discovery of MCV, three new polyomaviruses infecting skin, HPyV6, HPyV7, and TSV (trichodysplasia spinulosa-associated polyomavirus) (49, 54), and a ninth polyomavirus from the blood of immunosuppressed patients, HPyV9, were discovered (50). To date, these four additional viruses, with the possible exception of TSV and its associated proliferative skin disorder termed trichodysplasia spinulosa (35), have not yet been linked to human disease.

Unbiased DNA sequencing is rapidly becoming the method of choice for pathogen discovery, as high-throughput or “deep” sequencing of clinical samples facilitates the identification of novel, highly divergent pathogens that would elude detection by conventional PCR assays (18, 53). Previously, it has been shown that by shotgun sequencing as few as 1 million reads per clinical sample, sensitivities of detection comparable to PCR (<100 copies per mL) can be achieved for both known and candidate novel viruses (28).

### BRIEF SUMMARY OF THE INVENTION

The present invention is based on Applicants' discovery of a novel polyomavirus, provisionally named MX polyomavirus (MXPyV), first isolated from diarrheal stool collected from a child in Mexico. Subsequent PCR screening of stool samples revealed that MXPyV has a broad geographic prevalence. Detection of this polyomavirus may be used for diagnosis, surveillance, or prognosis determination of certain cancers or gastrointestinal illnesses, e.g. gastrointestinal cancer, ulcerative colitis, Crohn's disease, celiac disease, aiding the treatment of immunocomrpomised individuals, and identifying individuals afflicted with GI disorders or cancers and carriers of a genetic disorder that heightens susceptibility to such GI disorders or cancers.

Accordingly, the claimed subject matter provides compositions and methods useful in the detection, treatment and prevention, and modulation of MXPyV infection.

In one aspect, the invention provides an isolated nucleic acid comprising a nucleotide sequence of at least 100 nucleotides in length, the sequence having at least 90% sequence identity to a portion of the same length in SEQ ID NO:1 or its complement, wherein said sequence excludes that of polyomavirus MWPyV (St. Louis strain) and HPyV10. In some embodiments, the nucleotide sequence has at least 95% identity over its length to SEQ ID NO:1. In some embodiments, the nucleotide sequence has at least 90% identity over the full length of SEQ ID NO:1. In some embodiments, the nucleotide sequence has at least 95% identity over the full length of SEQ ID NO:1. In some embodiments, the nucleotide sequence comprises SEQ ID NO:1.

In another aspect, the invention provides an isolated expression vector comprising a nucleic acid disclosed herein.

In another aspect, the invention provides an isolated host cell comprising an expression vector disclosed herein. In some embodiments, the host cell is not a natural host cell for MXPyV. In still other embodiments, the host cell is a recombinant host cell. In other embodiments, the host cell is a non-human host cell.

In another aspect, the invention provides an isolated nucleic acid comprising a nucleotide sequence of at least 100 nucleotides in length, the sequence having at least 90% sequence identity to an open reading frame selected from the group consisting of SEQ ID NOs:2-7. In some embodiments, the nucleotide sequence has at least 95% identity to the open reading frame selected from the group consisting of SEQ ID NOs:2-7. In some embodiments, the nucleotide sequence comprises an open reading frame selected from the group consisting of SEQ ID NOs:2-7.

In another aspect, the invention provides an isolated peptide encoded by a nucleic acid sequence disclosed herein.

In another aspect, the invention provides an isolated antibody that specifically binds to a peptide disclosed herein. In some embodiments, the antibody is a polyclonal antibody. In some embodiments, the antibody is a monoclonal antibody.

In another aspect, the invention provides a method for detecting a polyomavirus in a biological sample, the method comprising the steps of:

(a) contacting the biological sample with a primer that hybridizes to a nucleotide sequence selected from SEQ ID NO:1-7;
(b) performing a nucleic acid amplification reaction to generate an amplicon; and
(c) detecting the amplicon with a probe that hybridizes under stringent hybridization conditions with SEQ ID NO: 1-7.

In another aspect, the invention provides a method for detecting a polyomavirus in a biological sample, the method comprising the steps of:
(a) contacting the biological sample with an antibody disclosed herein; and
(b) detecting the binding of the antibody to its target antigen in the biological sample,
   wherein the antibody is immobilized on a solid phase and the presence of the binding is indicative of polyomavirus in the biological sample.

In another aspect, the invention provides a method for detecting an anti-MXPyV antibody in a human biological sample, the method comprising the steps of:
(a) contacting the sample suspected of containing an anti-MXPyV antibody with an immobilized peptide described herein for a time and under conditions sufficient to allow the formation of a complex of the anti-MXPyV antibody with said peptide;
(b) adding a conjugate for a time and under conditions sufficient to allow the conjugate to bind to the anti-MXPyV antibody of the complex, the conjugate comprising an anti-human antibody attached to a signal generating compound capable of generating a detectable signal; and
(c) detecting the presence of the anti-MXPyV antibody in the sample by detecting the signal generated by the signal generating compound.

In still another aspect, the invention provides a method for detecting an anti-MXPyV antibody in a human biological sample, the method comprising the steps of:
(a) contacting the sample suspected of containing an anti-MXPyV antibody with a with an immobilized anti-human antibody, under time and conditions sufficient to allow the formation of a complex of the anti-MXPyV antibody with said immobilized antibody;
(b) adding a conjugate for a time and under conditions sufficient to allow the conjugate to bind to the anti-MXPyV antibody of the complex, the conjugate comprising a peptide described herein being attached to a signal generating compound capable of generating a detectable signal; and
(c) detecting the presence of the anti-MXPyV antibody in the sample by detecting the signal generated by the signal generating compound.

In still another aspect, the invention provides a method for detecting an anti-MXPyV antibody in a human biological sample, the method comprising the steps of:
(a) contacting the sample suspected of containing the anti-MXPyV antibody with an immobilized anti-human antibody, under time and conditions sufficient to allow the formation of a complex of the anti-MXPyV antibody with said immobilized antibody;
(b) adding a peptide described herein for a time and under conditions sufficient to allow the peptide to bind to the anti-MXPyV antibody of the complex;
(c) adding a conjugate for a time and under conditions sufficient to allow the conjugate to bind to peptide bound to the anti-MXPyV of the complex, the conjugate comprising a recombinant anti-MXPyV antibody attached to a signal generating compound capable of detecting a detectable signal; and
(d) detecting the presence of the anti-MXPyV in the sample by detecting the signal generated by the signal-generating compound.

In another aspect, the invention provides an immunogenic composition comprising an isolated peptide disclosed herein.

In another aspect, the invention provides a kit comprising at least one primer that hybridizes to a nucleotide sequence comprising SEQ ID NO:1.

In another aspect, the invention provides a kit comprising an antibody disclosed herein.

In still another aspect, the invention provides a kit comprising a MXPyV antigen disclosed herein.

### BRIEF DESCRIPTION OF THE DRAWINGS

FIG. 1A-B shows the genome organization of MXPyV. The 4,939-nt circular genome of MXPyV (A) contains coding regions for VP1, VP2, VP3, ST-Ag, and LT-Ag (yellow arrows). C1, C2, and C3 (gray) denote de novo assembled contigs from deep sequencing data. (B) Domains and binding motifs present in the spliced LT-Ag and ST Ag of MXPyV.

FIG. 2A-D shows an amino acid phylogenetic analysis of MXPyV relative to other polyomaviruses. (A) VP1, (B) VP2, (C) ST-Ag, (D) LT-Ag. Bayesian support levels are indicated at each branching point. Abbreviations: AGM, African green monkey; SV40, simian virus 40; SV12, simian virus 12; SqMPy, squirrel monkey; CaliSeaLion, California sea lion. Other abbreviations are described in the text. Note that Merkel cell virus (MCV) is not included in the LT-Ag phylogeny due to the presence of truncation mutations.

FIG. 3 is the MXPyV sequence. The entire MXPyV sequence is provided with identification of open reading frames.

FIG. 4 shows the whole-genome sequence alignment of MXPyV relative to other recently described gut-associated polyomaviruses HPyV10 and MWPyV.

### DETAILED DESCRIPTION OF THE INVENTION

Described herein is the identification and sequencing of the entire genome of a novel, highly divergent polyomavirus by deep sequencing of diarrheal samples. In accordance with the two-letter designations for human polyomaviruses, this virus has been provisionally named MX polyomavirus (MXPyV), after the country from which the initial isolate was identified. The ˜5.0 kb viral genome exhibits little overall homology (<46% amino acid identity) to known polyomaviruses, and due to phylogenetic variation among its individual proteins, cannot be placed in any existing taxonomic group.

The genomic organization and amino acid sequence homology of MXPyV, as well as conservation of known protein motifs in the T-antigen, indicate that this virus is indeed a polyomavirus. MXPyV is broadly distributed and was recovered from diarrheal samples from two continents. Furthermore, MXPyV isolates from different individuals showed sequence variation of 0-4.3%, and the virus was detected in children from birth to 6 years of age. Taken together, these findings strongly suggest that humans are the natural host, although growth of the virus in culture and serologic studies are needed for definitive confirmation.

By phylogenetic analysis, MxPyV does not consistently cluster with any other polyomavirus taxonomic group and, indeed, whereas MXPyV ORFs encoding VP1 and the large T-antigen cluster with some of the recently discovered human polyomaviruses (WU, KI, HPyV6, and HPyV7), VP2, MXPyV appears to group better with the rodent polymaviruses. In contrast, the small T-antigen of MXPyV does not appear to cluster with any of the known polyomavirus groups. These observations, combined with the low amino acid identity of 13-44% in the proteins of MXPyV relative to those of other polyomaviruses (Table 1), suggest that the ancestral strain for MXPyV likely diverged early along the evolutionary pathway, and raises the possibility of recombination of polyomavirus genes. Although recombination in polyomaviruses remains controversial, it does appear to occur, at least in JC viruses (16). No evidence for recombination within individual genes was detected by bootscanning analysis (data not shown), but this is to be expected given the high sequence divergence of MXPyV and absence of closely related phylogenetic neighbors. As the phylogenetic tree of polyomaviruses is currently sparse, with fewer than 30 members, it is likely that MxPyV, situated on a highly divergent branch, represents the first member of a new subclade of polyomaviruses.

Unlike other recently discovered polyomaviruses, which were found in respiratory secretions or cutaneous tissue, detection of MXPyV appears largely confined to stool, with a prevalence of 3.4% in fecal samples collected from California, Mexico, and Chile (Table 2), although one respiratory sample out of 136 (0.74%) also tested positive. SV40, BKV, JCV, and MCV have also been detected in human feces (38, 55, 56), although their primary sites of pathology are elsewhere in the human body, as have polyomaviruses WU and KI (4, 5, 46). Thus, although the detection of MXPyV in stool and rarely in respiratory secretions (Table 2) is consistent with the hypothesis of a general fecal-oral route for polyomavirus transmission (55), the gastrointestinal tract may not be the primary tissue reservoir for MXPyV. MXPyV was not detectable in 480 plasma/urine samples from highly immunocompromised transplant recipients, indicating that these are not reservoir sites for MXPyV infection, as is the case for JC and BK viruses. Further investigation of MXPyV in both healthy and diseased individuals is needed to determine if there are indeed tissue reservoirs for MXPyV in humans.

No association between MXPyV infection and diarrhea was detected in the California and Chile gastroenteritis studies for which controls were available (Tables 2 and 3). In fact, in the samples from Chile, the trend was reversed, with 4 MXPyV367 positive samples among 96 asymptomatic control individuals and no positives among 96 children with diarrhea (Table 1). These findings, however, do not preclude the possibility of MXPyV as an etiologic agent of diarrhea given the low prevalence rate of 3.4% in stool samples and the fact that a large proportion of infections from diarrheal viruses are asymptomatic (7, 39). Notably, 6 of 12 MXPyV372 positive diarrheal samples from Mexico tested negative by a broad-spectrum viral microarray and specific PCR assays for all known diarrheal viruses (Supplementary Table 1), suggesting that MXPyV may still potentially be a cause of gastroenteritis. Serologic testing before and after diarrheal episodes would be useful in investigating this possibility, as shown previously for a human cardiovirus and klassevirus/salivirus (13, 29).

In the California SIFT study, a significantly increased number of MXPyV infections was seen in girls than in boys (13 female vs. 4 males, p=0.012) (Table 4). This observation is intriguing in light of the fact that apparent gender differences have previously been described in a serological investigation of primary infections by Merkel cell virus (MCV) in childhood (11). In that study, males showed higher seroconversion and seroprevalence rates to MCV than females. This apparent gender difference was not observed with respect to MCV seroprevalence in adults (36), although gender does appear to dramatically impact incidence and survival rates associated with Merkel cell carcinoma (2, 3). Whether differences in the age at which MXPyV is acquired, childhood physiology, or viral characteristics play a role in the gender differences observed here is unknown, and merits further investigation.

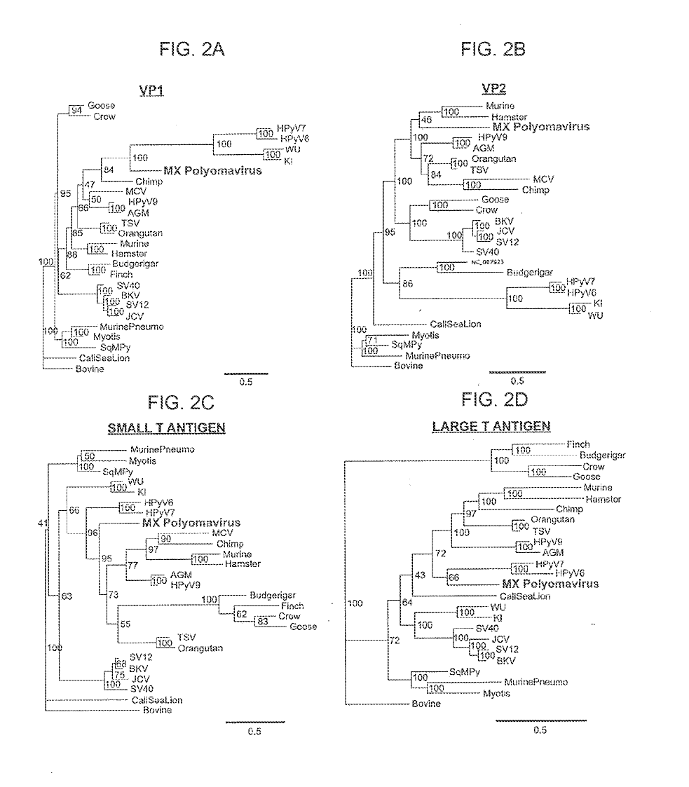

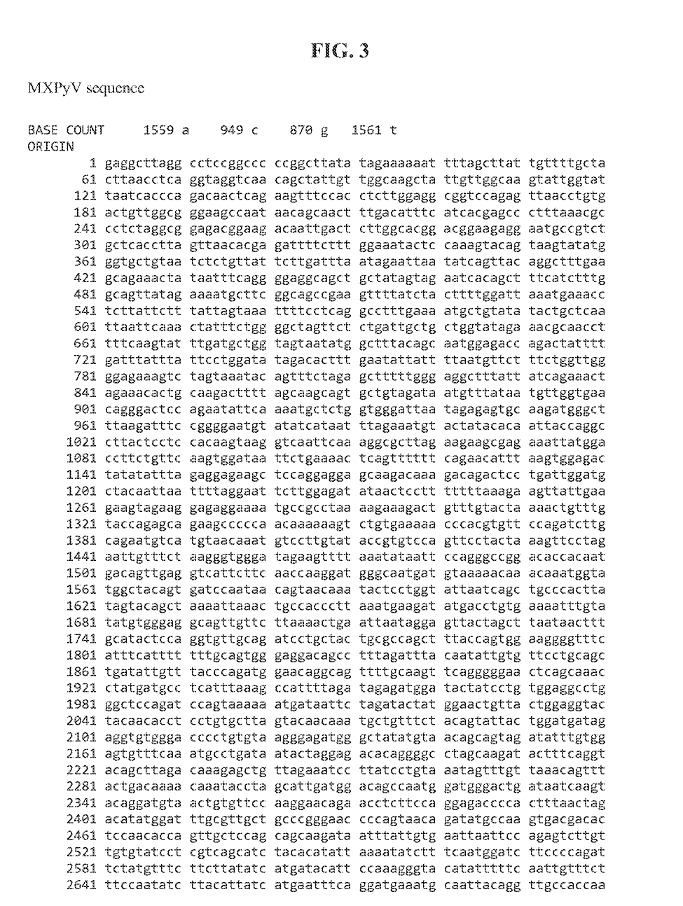

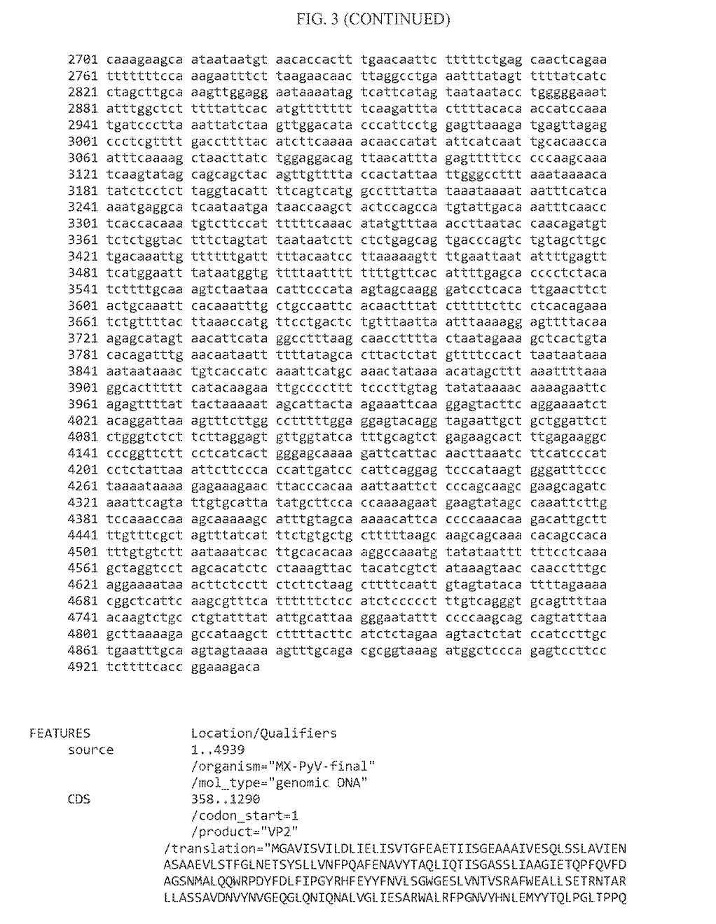

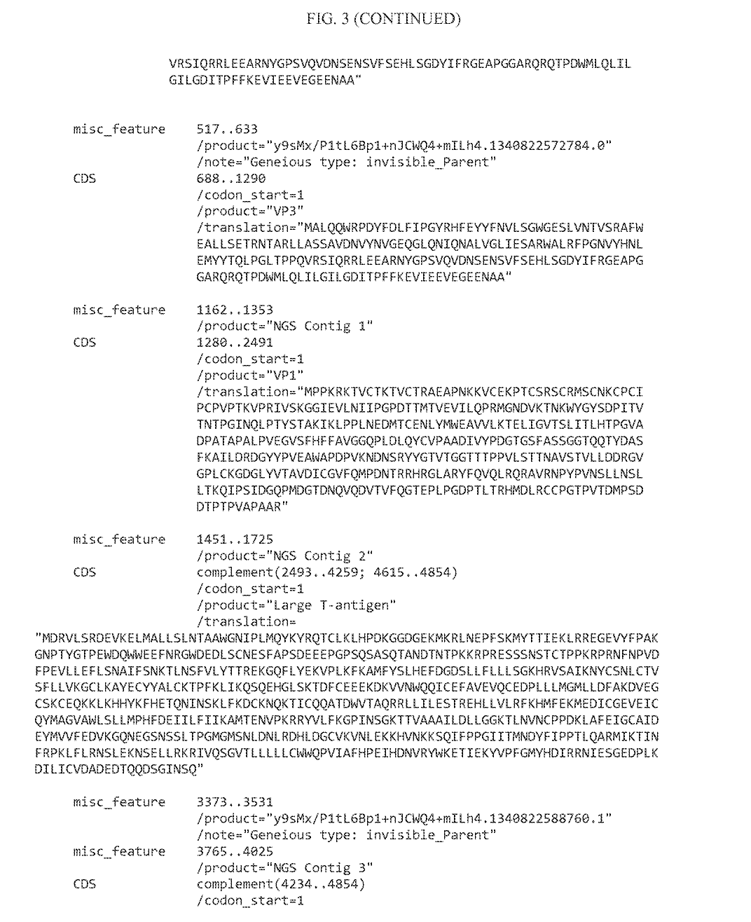

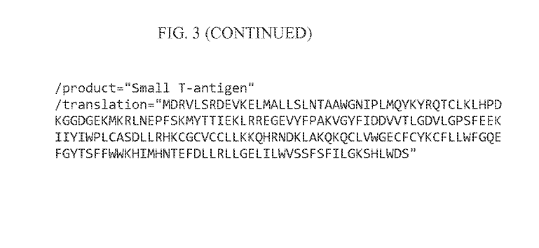

The full extent of pathology associated with MXPyV remains to be elucidated. Given the discovery and detection of MxPyV in stool and the greater incidence of latent polyomavirus infections in immunocompromised individuals, it may be worthwhile to continue searching for MXPyV in transplant patients suffering from unexplained diarrhea or other gastrointestinal illnesses. In addition, as MXPyV retains conserved CR1, DnaJ, pRB1-binding, and PP2A domains (FIG. 2A) previously demonstrated to play roles in virus induced cell transformation (17, 42, 43, 57), it is plausible that MXPyV may be involved in tumorigenesis, a possibility that can now be formally explored by specific molecular testing for the virus in cancer tissues. The detection of MXPyV in a child at the time of an acute gastroenteritis episode and 3 months later does suggest that, in analogy with other human polyomaviruses (27), persistent infection by MXPyV can occur and thus potentially play a role in the development of chronic diseases such as cancer.

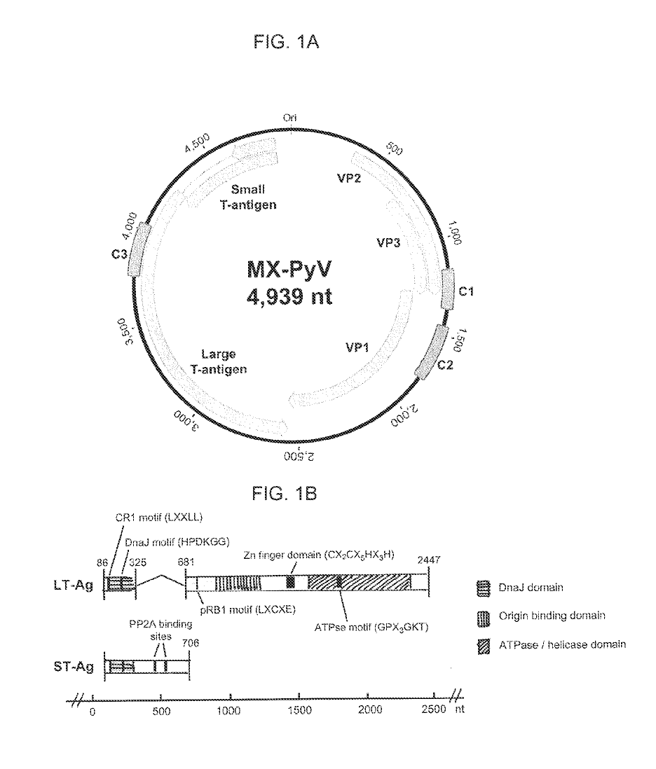

The discovery of the novel polyomavirus MXPyV further establishes deep sequencing as a powerful method to identify candidate pathogens, both known and novel, in clinical samples. Notably, despite the low overall amino acid sequence identity of MXPyV relative to known polyomaviruses, three separate regions from MxPyV were identifiable from short, 100-bp deep sequencing reads, including a recovered contig that precisely overlaps the conserved origin binding domain of the LT-Ag (FIGS. 1A, “C3” and 1B, “origin binding domain”). With the advent of even longer deep sequencing read lengths that are critical for novel pathogen identification and routine yields exceeding a billion reads per run, it is now possible to explore the human virome in both healthy and diseased states at an unprecedented depth.

Provided are compositions and methods useful for the identification, isolation, expression, purification, detection, treatment, preventions, and modulation of MXPyV.

### DEFINITIONS

Unless otherwise noted, the technical terms used herein are according to conventional usage as understood by persons skilled in the art. Definitions of common terms in molecular biology may be found in standard texts (e.g. Benjamin Lewin, Genes V, published by Oxford University Press, 1994 (ISBN 0-19854287-9); Kendrew et al. (eds.), The Encyclopedia of Molecular Biology, published by Blackwell Science Ltd, 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8)).

The term “nucleic acid” refers to deoxyribonucleotides or ribonucleotides and polymers thereof in either single- or double-stranded form, and complements thereof. Unless otherwise indicated, a particular nucleic acid sequence also implicitly encompasses conservatively substituted variants thereof (e.g., degenerate codon substitutions) and complementary sequences, as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide. Unless other specified, a particular nucleotide sequence may also encompasses “splice variants,” which as the name suggests are products of alternative splicing of a gene. After transcription, a pre-mRNA can be spliced such that the exons of the pre-mRNA are spliced together in different combinations, generating two or more different mature mRNAs from a single pre-mRNA, which in in turn may encode different polypeptides.

The term “isolated” as used herein with respect to nucleic acids, such as DNA or RNA, refers to molecules separated from other DNAs or RNAs, respectively, that are present in the natural source of the macromolecule. Isolated is meant to include nucleic acid fragments which are not naturally occurring as fragments. The term isolated as used herein also refers to a nucleic acid or peptide that is substantially free of cellular material, viral material, or culture medium when produced by recombinant DNA techniques, or chemical precursors, or other chemicals when chemically synthesized.

“Percent sequence identity,” “percent amino acid sequence identity,” “percent gene sequence identity,” and/or “percent nucleic acid/polynucleotide sequence identity,” with respect to two amino acids, polynucleotide and/or gene sequences (as appropriate), refer to the percentage of residues that are identical in the two sequences when the sequences are optimally aligned. Thus, 80% amino acid sequence identity means that 80% of the amino acids in two optimally aligned polypeptide sequences are identical. Sequence identity is determined using standard techniques known in the art (see e.g., Smith and Waterman, Adv Appl Math, 2:482, 1981; Needleman and Wunsch, J Mol Biol, 48:443, 1970; Pearson and Lipman, Proc Natl Acad Sci USA, 85:2444, 1988; programs such as BLAST, ALIGN, CLUSTAL, GAP, BESTFIT, FASTA, and TFASTA in the Wisconsin Genetics Software Package, Genetics Computer Group, Madison, Wis.; and Devereux et al., Nucl Acid Res, 12:387-395, 1984).

The phrase “substantially identical” in the context of two nucleic acids or polypeptides thus refers to a polynucleotide or polypeptide that comprising at least 70% sequence identity, preferably at least 75%, preferably at least 80%, preferably at least 85%, preferably at least 90%, preferably at least 95%, preferably at least 97%, preferably at least 98% and preferably at least 99% sequence identity as compared to a reference sequence using the programs or algorithms (e.g., BLAST, ALIGN, CLUSTAL) operating with standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted (e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J MoL Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“MX polyomavirus” or “MXPyV” refers to both the genetic components of the virus, e.g., the DNA and RNA transcripts thereof, proteins encoded by the genome (including structural and nonstructural proteins), and viral particles.

Proteins or “MXPyV antigens” of the present invention will satisfy one or more of the following characteristics: (1) structural and non-structural MX polyomavirus proteins encoded by nucleic acids that have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or 100% sequence identity, to a region of at least about 25, 50, 100, 200, 500, 1000 or more nucleic acids, up to the full length sequence of SEQ ID NO:1; (2) proteins that specifically bind to antibodies, e.g., polyclonal or monoclonal antibodies, raised against an immunogen comprising an amino acid sequence of a protein encoded by an open reading frame of SEQ ID NOs:2-7; and conservatively modified variants thereof; (3) proteins encoded by nucleic acids that specifically hybridize under stringent hybridization conditions to an anti-sense strand corresponding to a nucleic acid sequence of SEQ ID NOs:2-7; and (4) proteins having greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000 or more amino acids, to a protein encoded by an open reading frame of SEQ ID NOs:2-7.

The term “open reading frame” or “ORF” refers to a length of DNA or RNA sequence capable of being translated into a peptide normally located between a start or initiation signal and a termination signal.

The term “expression vector” indicates a plasmid, a virus or another medium, known in the art, into which a nucleic acid sequence for encoding a desired protein can be inserted or introduced.

The term “host cell” is a cell that is susceptible to transformation, transfection, transduction, conjugation, and the like with a nucleic acid construct or expression vector. Host cells can be derived from plants, bacteria, yeast, fungi, insects, animals, etc.

The terms “polypeptide” or “peptide” or “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Macromolecular structures such as polypeptide structures can be described in terms of various levels of organization. For a general discussion of this organization (see, e.g., Alberts et al., Molecular Biology of the Cell (3^{rd} ed., 1994) and Cantor and Schimmel, Biophysical Chemistry Part I: The Conformation of Biological Macromolecules (1980)). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity. Typical domains are made up of sections of lesser organization such as stretches of 3-sheet and a-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes Amino acid substitutions, deletions or additions to individual or a small percentage of amino acids in the encoded sequence is a conservatively modified variant, where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “antibody” refers to a polypeptide comprising a framework region from an immunoglobulin gene or fragments thereof that specifically binds and recognizes an antigen. The term “antigen” refers to any molecule capable of being bound by an antibody or a T cell receptor if presented by MHC molecules. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VI) and variable heavy chain (V_{H}) refer to these light and heavy chains respectively. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 bp a disulfide bond. The F(ab)′2 can be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments can be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies that are raised to MXPyV antigens, polymorphic variants, alleles, orthologs, and conservatively modified variants, or splice variants, or portions thereof, can be selected to obtain only those polyclonal antibodies that are specifically immunoreactive with MXPyV antigens and not with other proteins. This selection can be achieved by subtracting out antibodies that cross-react with other molecules. A variety of immunoassay formats can be used to select antibodies specifically immunoreactive with a particular protein, as described herein.

The term “detectable moiety” or “conjugate” refers to any atom, molecule or a portion thereof, the presence, absence or level of which is directly or indirectly monitorable. A variety of detectable moieties are well known to those skilled in the art, and can be any material detectable by spectroscopic, photochemical, biochemical, immunochemical, electrical, optical or chemical means. Such detectable labels can include, but are not limited to, magnetic beads, fluorescent dyes, radiolabels, enzymes, and colorimetric labels such as colloidal gold or colored glass or plastic beads, each of which is described in greater detail herein.

The term “vaccine” refers to a pharmaceutical composition comprising at least one immunologically active component that induces an immunological response in an animal and possibly but not necessarily one or more additional components that enhance the immunological activity of the active component. A vaccine can additionally comprise further components typical to pharmaceutical compositions. The immunologically active component of a vaccine can comprise complete virus particles in either their original form or as attenuated particles in a so called modified live vaccine (MLV) or particles inactivated by appropriate methods in a so called killed vaccine (KV). A vaccine comprising antigenic substances can be administered for the purpose of inducing a specific and active immunity against a disease provoked by a MXPyV infection. A vaccine can also provide passive immunity in the form of antibodies previously generated against MXPyV antigens.

The term “immune response” or “immunological response” refers to a reaction of the immune system to an antigen in the body of a host, which includes generation of an antigen-specific antibody and/or cellular cytotoxic response. The term further refers to an immune system response that leads to a condition of induced sensitivity to an immunogeneic product.

A “biological sample” or “sample” includes sections of tissues such as biopsy and autopsy samples, and frozen sections taken for histologic purposes. Such samples include stool, blood and blood fractions or products (e.g., serum, plasma, platelets, red blood cells, and the like), tissue (e.g. cancer tissue), sputum, cloacal swabs, mucosa, cultured cells, e.g., primary cultures, explants, and transformed cells, biological fluids, urine, etc. A biological sample is typically obtained from a eukaryotic organism. The tissue sampled can be, for instance, skin, brain (e.g., cerebrum, cerebellum, optic lobe), spinal cord, adrenals, pectoral muscle, lung, heart, liver, crop, proventriculus, ventriculus, duodenum, small intestine, large intestine, cloaca, kidney, bursa of fabricus, spleen, pancreas, adrenal gland, bone marrow, lumbosacral spinal cord, or blood. Contacting a sample refers to the plain and ordinary meaning to refer to exposing the sample.

The term “detecting,” when in reference to detecting the presence of MXPyV, refers to determining the presence, using any method, of the virus or viral particles including viral antigens, inside cells, on cells, and/or in medium with which cells or the virus have come into contact. The methods are exemplified by, but not limited to, the observation of cytopathic effect, detection of viral protein, such as by immunofluorescence, ELISA, or Western blot hybridization, detection of viral nucleic acid sequence, such as by PCR, RT-PCR, Southern blots, and Northern blots, nucleic acid hybridization, nucleic acid arrays, and the like.

The phrase “MXPyV infection” refers to the invasion by, multiplication and/or presence of MXPyV in a cell or a subject with or without symptoms.

The phrase “functional effect” in the context of assays for testing compounds that modulate activity of MXPyV, or for treating or preventing MXPyV infection, includes the determination of a parameter that is indirectly or directly under the influence of MXPyV, e.g., a phenotypic or chemical effect, such as the ability to increase or decrease viral genome replication, viral RNA and protein production, virus packaging, viral particle production (particularly replication competent viral particle production), cell receptor binding, viral transduction, cellular infection, antibody binding, inducing a cellular or humoral immune response, viral protein enzymatic activity, etc. “Functional effects” include in vitro, in vivo, and ex vivo activities. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for a protein; measuring inducible markers or transcriptional activation of a protein; measuring binding activity or binding assays, e.g. binding to antibodies; measuring changes in ligand or substrate binding activity; measuring viral replication; measuring cell surface marker expression; measurement of changes in protein levels; measurement of RNA stability; identification of downstream or reporter gene expression (CAT, luciferase, 0-gal, GFP and the like), e.g., via chemiluminescence, fluorescence, colorimetric reactions, antibody binding, and inducible markers.

The term “test compound” or “compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis. Compounds can be immune modulators, e.g. inhibitors, activators, with respect to MXPyV. Inhibitors are compounds that, e.g., bind to, partially or totally block activity, decrease, prevent, delay activation, inactivate, desensitize, or down regulate the activity or expression of MXPyV, e.g., antagonists. Activators are compounds that increase, open, activate, facilitate, enhance activation, sensitize, agonize, or up regulate MXPyV activity, e.g., agonists Inhibitors, activators, or modulators also include genetically modified versions of MXPyV, e.g., versions with altered activity, as well as naturally occurring and synthetic ligands, substrates, antagonists, agonists, antibodies, peptides, cyclic peptides, nucleic acids, antisense molecules, ribozymes, or small chemical molecules for example.

The phrase “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

The term “aptamer” refers to a non-naturally occurring nucleic acid having a desirable action on a target. A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. Aptamer action can be specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule.

An “siRNA” molecule or an “RNAi” molecule refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length. See also PCT/US03/07237, herein incorporated by reference in its entirety.

The term “antisense” refers to an oligomeric compound or molecule that is at least partially complementary to a target nucleic acid molecule to which it hybridizes. Antisense compounds or molecules can include, but are not limited to, oligonucleotides, oligonucleosides, oligonucleotide analogs, oligonucleotide mimetics, and chimeric combination.

An siRNA or antisense molecule or RNAi molecule is “specific” for a target nucleic acid if it reduces expression of the nucleic acid by at least about 10% when the siRNA or RNAi is expressed in a cell that expresses the target nucleic acid.

The term “treating” or “treatment” includes the application or administration of a composition to a subject, or application or administration of a composition to a cell or tissue from a subject who has been infected with MXPyV, or has symptoms of MXPyV infection, with the purpose of curing, healing, alleviating, relieving, altering, remedying, ameliorating, improving, or affecting the disease or condition, the symptom of the disease or condition, or the risk of the disease or condition.

The term “preventing” or “prevention” includes stopping or hindering a disease, disorder, or symptoms associated with MXPyV infection.

As used herein, the term “subject” or “individual” includes any human or non-human animal. The term “nonhuman animal” includes all vertebrates, e.g. mammals and non-mammals, such as nonhuman primates, sheep, dogs, cats, horses, cows, chickens, amphibians, reptiles, etc.

The term “administering” or “administration” refers to therapeutically or prophylactically administering an effective amount of a composition or medicament during the course of therapy. Prophylactic administration can occur prior to manifestation of symptoms characteristic of a MXPyV infection.

The phrase “therapeutically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The phrase “stringent conditions” refers to conditions under which a probe will hybridize to its target subsequence, typically in a complex mixture of nucleic acids, but to no other sequences. The term “hybridize” refers to the process by which single strands of nucleic acid sequences form double-helical segments through hydrogen bonding between complementary nucleotides. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH.

Tₘ is the temperature (under defined ionic strength, pH, and nucleic concentration) at which 50% of the probes complementary to the target hybridize to the target sequence at equilibrium (as the target sequences are present in excess, at T, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference (e.g., and Current Protocols in Molecular Biology, ed. Ausubel, et al.).

### Isolation, Expression, Purification, and Detection of MXPyV

The subject matter described herein relies on routine techniques in the field of recombinant genetics. Recombinant when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

Basic texts disclose general methods of use in this invention (e.g. Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

### MXPyV Expression

To obtain high level expression of a cloned gene or genome, one typically subclones the nucleic acid into an expression vector that contains a strong promoter to direct transcription, a transcription/translation terminator, and if for a nucleic acid encoding a protein, a ribosome binding site for translational initiation. Suitable bacterial promoters are well known in the art and described (e.g., in Sambrook et al., and Ausubel et al, supra. Bacterial expression systems for expressing the protein are available in, e.g., E. coli, Bacillus sp., and Salmonella (Palva et al., Gene 22:229-235 (1983); Mosbach et al., Nature 302:543-545 (1983)). Kits for such expression systems are commercially available. Eukaryotic expression systems for mammalian cells, yeast, and insect cells are well known in the art and are also commercially available. Retroviral expression systems can be used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. Heterologous refers to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the nucleic acid of choice and signals required for efficient polyadenylation of the transcript, ribosome binding sites, and translation termination. Additional elements of the cassette can include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region can be obtained from the same gene as the promoter sequence or can be obtained from different genes.

The particular expression vector used to transport the genetic information into the cell is not particularly critical. Any of the conventional vectors used for expression in eukaryotic or prokaryotic cells can be used. Standard bacterial expression vectors include plasmids such as pBR322, pSKF, pET23D, and fusion expression systems such as MBP, GST, and LacZ. Epitope tags can also be added to recombinant proteins to provide convenient methods of isolation, e.g., c-myc. Sequence tags can be included in an expression cassette for nucleic acid rescue. Markers such as fluorescent proteins, green or red fluorescent protein, 13-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMT010/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can also be regulated using inducible promoters. With inducible promoters, expression levels are tied to the concentration of inducing agents, such as tetracycline, by the incorporation of response elements for these agents into the promoter. Generally, high level expression is obtained from inducible promoters only in the presence of the inducing agent; basal expression levels are minimal.

Vectors can have a regulatable promoter, e.g., tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a sequence of choice under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, as any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J Bact. 132:349-351 (1977); Clark-Curtiss & Curtiss, Methods in Enzymology 101:347-362 (Wu et al., eds, 1983)).

Any of the well-known procedures for introducing foreign nucleotide sequences into host cells can be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing MXPyV proteins and nucleic acids.

After the expression vector is introduced into the cells, the transfected cells are cultured under conditions favoring expression of the protein of choice, which is recovered from the culture using standard techniques identified below.

Either naturally occurring or recombinant MXPyV proteins can be purified for use in diagnostic assays, for making antibodies (for diagnosis and therapy) and vaccines, and for assaying for anti-viral compounds. Naturally occurring protein can be purified, e.g., from primate tissue samples. Recombinant protein can be purified from any suitable expression system.

### MXPyV Proteins

The protein can be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant protein is being purified. For example, proteins having established molecular adhesion properties can be reversible fused to the protein. With the appropriate ligand or substrate, a specific protein can be selectively adsorbed to a purification column and then freed from the column in a relatively pure form. The fused protein is then removed by enzymatic activity. Finally, protein could be purified using immunoaffinity columns. Recombinant protein can be purified from any suitable source, include yeast, insect, bacterial, and mammalian cells.

Recombinant proteins can be expressed and purified by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria can form insoluble aggregates (“inclusion bodies”). Several protocols are suitable for purification of protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies can be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation can occur upon removal (by dialysis, for example) or dilution of the denaturant, allowing re-formation of immunologically and/or biologically active protein. Other suitable buffers are known to those skilled in the art. Human proteins are separated from other bacterial proteins by standard separation techniques, e.g., with Ni-NTA agarose resin.

Alternatively, it is possible to purify recombinant protein from bacteria periplasm. After lysis of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO4 and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Solubility fractionation can be used as a standard protein separation technique for purifying proteins. As an initial step, particularly if the protein mixture is complex, an initial salt fractionation can separate many of the unwanted host cell proteins (or proteins derived from the cell culture media) from the recombinant protein of interest. The preferred salt is ammonium sulfate Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The protein can also be separated from other proteins on the basis of its size, net surface charge, hydrophobicity, and affinity for ligands or substrates using column chromatography. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### Detecting the Presence or Absence of MXPyV

Described herein are diagnostic assays to detect MXPyV, MXPyV nucleic acids (genome and genes), MXPyV antibodies in an infected subject, and MXPyV proteins.

### Detecting MXPyV Nucleic Acids

MXPyV infection can be detected based on the level of a MXPyV RNA or DNA in a biological sample. One may use the nucleic acid sequences of the present invention to synthesize DNA oligomers of about 8 10 nucleotides, or larger, which are useful as hybridization probes in detect the presence of the viral genome in, for example, the sera of subjects suspected of harboring the MXPyV virus or for screening donated blood for the presence of the virus. The nucleic acid sequences of the present invention also allow for the design and production of MXPyV-specific polypeptides which may be used as diagnostic reagents for the presence of antibodies raised during infection with the virus.

Primers may also be developed using the nucleic acid sequences of the present invention. Such primers can be used for detection of MXPyV, diagnosis, and determination of MXPyV viral load. Any suitable primer can be used to detect the genome, nucleic acid sub sequence, ORF, or protein of choice, using, e.g., methods described in US 20030104009. For example, the subject nucleic acid compositions can be used as single- or double-stranded probes or primers for the detection of MXPyV mRNA or cDNA generated from such mRNA, as obtained can be present in a biological sample (e.g., extracts of human cells). The MXPyV polynucleotides of the invention can also be used to generate additional copies of the polynucleotides, to generate antisense oligonucleotides, and as triple-strand forming oligonucleotides. For example, two oligonucleotide primers can be employed in a polymerase chain reaction (PCR) based assay to amplify a portion of MXPyV cDNA derived from a biological sample, wherein at least one of the oligonucleotide primers is specific for (i.e., hybridizes to) the MXPyV polynucleotide. The primers are preferably at least or about 12, 15, 16, 18, 20, 22, 24, 25, 30, 35, 40, 45, or 50 nt or are, for instance, from about 12 to 50 nt in length, 15 to 30 nt in length, 15 to 25 nt in length, or 20 to 30 nt in length) fragments of a contiguous sequence of SEQ ID NO: 1 or other polynucleotide sequence encoding an MXPyV nucleic acid or polypeptide. The amplified cDNA is then separated and detected using techniques well known in the art, such as gel electrophoresis. Similarly, oligonucleotide probes that specifically hybridize to a MXPyV polynucleotide can be used in a hybridization assay to detect the presence of the MXPyV polynucleotide in a biological sample.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided (e.g., in Innis et al. (1990) PCR Protocols, A Guide to Methods and Applications, Academic Press, Inc. N.Y.)).

Nucleic acid probes or primers specific to MXPyV can be generated using the polynucleotide sequences disclosed herein. The probes are preferably at least about 12, 15, 16, 18, 20, 22, 24, or 25 nt fragments of a contiguous sequence from SEQ ID NO: 1 or other polynucleotide sequence encoding a MXPyV nucleic acid or polypeptide. Nucleic acid probes can be less than about 200 bp, 150 bp, 100 bp, 75 bp, 50 bp, 60 bp, 40 bp, 30 bp, 25 bp 2 kb, 1.5 kb, 1 kb, 0.5 kb, 0.25 kb, 0.1 kb, or 0.05 kb in length. The probes can be produced by, for example, chemical synthesis, PCR amplification, generation from longer polynucleotides using restriction enzymes, or other methods well known in the art. Preferred primers and probes are identical to a MXPyV nucleic acid sequence that is distinguishable by hybridization assays from a non-MXPyV sequence.

The polynucleotides described herein, particularly where used as a probe in a diagnostic assay, can be detectably labeled. Exemplary detectable labels include, but are not limited to, radiolabels, fluorochromes, (e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,T,4,7-hexachlorofluorescein (HEX), 5-carboxyfluorescein (5-FAM) or N,N,N′,N′-tetramethyl-6-carboxyrho-damine (TAMRA)), radioactive labels, (e.g. sup.32p, .sup.35S, and sup.3H), and the like. The detectable label can involve two stage systems (e.g., biotin-avidin, hapten-anti-hapten antibody, and the like).

Non-PCR-based, sequence specific DNA amplification techniques can also be used with the invention to detect MXPyV sequences. An example of such techniques include, but is not necessarily limited to, the Invader assay (see, e.g., Kwiatkowski et al. Mol. Diagn. December 1999, 4:353-64. See also U.S. Pat. No. 5,846,717).

The claimed subject matter can also include solid substrates, such as arrays, comprising any of the polynucleotides described herein. The polynucleotides are immobilized on the arrays using methods known in the art. An array can have one or more different polynucleotides.

Any suitable qualitative or quantitative methods known in the art for detecting specific MXPyV nucleic acid (e.g., RNA or DNA) can be used. MXPyV nucleic acid can be detected by, for example, in situ hybridization in tissue sections, using methods that detect single base pair differences between hybridizing nucleic acid (e.g., using the Invader® technology described in, for example, U.S. Pat. No. 5,846,717), by reverse transcriptase-PCR, or in Northern blots containing poly A+ mRNA, and other methods well known in the art. For detection of MXPyV polynucleotides in blood or blood-derived samples, the use of methods that allow for detection of single base pair mismatches is preferred.

Using the MXPyV nucleic acid as a basis, nucleic acid probes (e.g., including oligomers of at least about 8 nucleotides or more) can be prepared, either by excision from recombinant polynucleotides or synthetically, which probes hybridize with the MXPyV nucleic acid, and thus are useful in detection of MXPyV virus in a sample, and identification of infected individuals, as well as further characterization of the viral genome(s). The probes for MXPyV polynucleotides (natural or derived) are of a length or have a sequence which allows the detection of unique viral sequences by hybridization. While about 6-8 nucleotides may be useful, longer sequences may be preferred, e.g., sequences of about 10-12 nucleotides, or about 20 nucleotides or more. Preferably, these sequences will derive from regions which lack heterogeneity among MXPyV viral isolates.

Nucleic acid probes can be prepared using routine methods, including automated oligonucleotide synthetic methods. A complement to any unique portion of the MXPyV genome will be satisfactory, e.g., a portion of the MXPyV genome that allows for distinguishing MXPyV from other viruses that may be present in the sample, e.g., other MXPyV. For use as probes, complete complementarity is desirable, though it can be unnecessary as the length of the fragment is increased.

For use of such probes as diagnostics, the biological sample to be analyzed, such as blood or serum, can be treated, if desired, to extract the nucleic acids contained therein. The resulting nucleic acid from the sample can be subjected to gel electrophoresis or other size separation techniques; alternatively, the nucleic acid sample can be dot blotted without size separation. The probes are usually labeled with a detectable label. Suitable labels, and methods for labeling probes are known in the art, can include, for example, radioactive labels incorporated by nick translation or kinasing, biotin, fluorescent probes, and chemiluminescent probes. The nucleic acids extracted from the sample are then treated with the labeled probe under hybridization conditions of suitable stringencies.

The probes can be made completely complementary to the MXPyV genome or portion thereof (e.g., to all or a portion of a sequence encoding a MXPyV small T-antigen). Therefore, usually high stringency conditions are desirable in order to prevent or at least minimize false positives. However, conditions of high stringency should only be used if the probes are complementary to regions of the viral genome which lack heterogeneity among MXPyV viral isolates. The stringency of hybridization is determined by a number of factors during hybridization and during the washing procedure, including temperature, ionic strength, length of time, and concentration of formamide (Sambrook et al. (1989), “Molecular Cloning; A Laboratory Manual,” Second Edition (Cold Spring Harbor Press, Cold Spring Harbor, N.Y.)).

Generally, it is expected that the MXPyV sequences will be present in a biological sample (e.g., stool, nasal secretion) obtained from an infected individual at relatively low levels, e.g., at approximately 10²-10⁴ MXPyV sequences per 10⁶ cells. This level can require that amplification techniques be used in hybridization assays. Such techniques are known in the art.

For example, the Enzo Biochemical Corporation “Bio-Bridge” system uses terminal deoxynucleotide transferase to add unmodified 3′-poly-dT-tails to a DNA probe. The poly dT-tailed probe is hybridized to the target nucleotide sequence, and then to a biotin-modified poly-A. PCT Publication No. WO84/03520 and European application no. EPAl24221 describe a DNA hybridization assay in which: (1) analyte is annealed to a single-stranded DNA probe that is complementary to an enzyme-labeled oligonucleotide; and (2) the resulting tailed duplex is hybridized to an enzyme-labeled oligonucleotide. EPA 204510 describes a DNA hybridization assay in which analyte DNA is contacted with a probe that has a tail, such as a poly-dT tail, an amplifier strand that has a sequence that hybridizes to the tail of the probe, such as a poly-A sequence, and which is capable of binding a plurality of labeled strands.

A particularly desirable technique can first involve amplification of the target MXPyV sequences in sera approximately 10,000 fold, e.g., to approximately 10 sequences/mL. This can be accomplished, for example, by the polymerase chain reactions (PCR) technique (Saiki et al. (1986), by Mullis, U.S. Pat. No. 4,683,195, and by Mullis et al. U.S. Pat. No. 4,683,202). Other amplification methods are well known in the art.

The probes, or alternatively nucleic acid from the samples, can be provided in solution for such assays, or can be affixed to a support (e.g., solid or semi-solid support). Examples of supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride, diazotized paper, nylon membranes, activated beads, and Protein A beads.

Probes (or sample nucleic acid) can be provided on an array for detection. Arrays can be created by, for example, spotting polynucleotide probes onto a substrate (e.g., glass, nitrocellulose, and the like) in a two-dimensional matrix or array. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described in EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734. Arrays are particularly useful where, for example a single sample is to be analyzed for the presence of two or more nucleic acid target regions, as the probes for each of the target regions, as well as controls (both positive and negative) can be provided on a single array. Arrays thus facilitate rapid and convenience analysis.

### MXPyV Antibodies

Antibodies raised against MXPyV can serve a wide variety of purposes, as described herein, which include, but are not limited to, diagnostic assays for the detection of MXPyV. A number of immunogens comprising portions of a MXPyV protein, virus or nucleic acid can be used to develop monoclonal and/or polyclonal antibodies which bind to the immunological epitope(s) of interest of MXPyV. Methods of producing such antibodies are well known to those of ordinary skill in the art (see, e.g., Kohler and Milstein, Nature 256:494 (1975), Mimms et al., Virology 176:604 619 (1990), Hammerling et al., Protein Purification, Principles and Practice, 2.sup.nd ed., Springer-Verlag, New York (1984)).

For example, a recombinant MXPyV protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein can also be used either in pure or impure form. The product is then injected into an animal capable of producing antibodies. Either monoclonal or polyclonal antibodies can be generated, for subsequent use in immunoassays to measure the protein.

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)).

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-MXPyV proteins and nucleic acids, using a competitive binding immunoassay. Specific polyclonal antisera and monoclonal antibodies will usually bind with a Kd of at least about 0.1 mM, more usually at least about 1 uM, preferably at least about 0.1 uM or better, and most preferably, 0.01 uM or better. Antibodies specific only for a particular MXPyV protein can also be made by subtracting out other cross-reacting proteins. In this manner, antibodies that bind only to the protein of choice can be obtained.

Phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Chimeric antibodies can be used, which is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Humanized or primatized antibodies can be used. Generally, a humanized antibody has one or more amino acid residues introduced into it from a source which is non-human. These non-human amino acid residues are often referred to as import residues, which are typically taken from an import variable domain. Methods for humanizing or primatizing non-human antibodies are well known in the art. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Once the specific antibodies against a MXPyV protein, virus or nucleic acid in are available, the antigen can be detected and/or quantified using any of a number of well recognized immunological binding assays (see, e.g., U.S. Pat. Nos. 4,366,241; 4,376,110; 4,517,288; and 4,837,168). MXPyV viral particles can be detected based on an epitope defined by the viral proteins as presented in a viral particle and/or an epitope defined by a viral protein that is separate from a viral particle. As used in this context, then, “antigen” is meant to refer to a MXPyV polypeptide as well as MXPyV viral particles. For a review of immunoassays, see also The Immunoassay Handbook, Third Edition (David Geoffrey Wild, ed., 3^{rd} ed. 2005) Elsevier Ltd., Oxford, UK; Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice. The antibody can be produced by any of a number of means well known to those of skill in the art and as described above.

### Immunoassays

As noted above, the present invention includes methods of detecting antibody to MXPyV using the viral protein or antigen peptides, e.g. VP1, VP2, VP3, ST-Ag, LT-Ag. More specifically, there are two basic types of assays, competitive and non-competitive (e.g., immunometric and sandwich). Each type can be either quantitative or non-quantitative. In both types of assays for solid phase embodiments, antibody or antigen reagents are covalently or non-covalently attached to the solid phase. Linking agents for covalent attachment are known and may be part of the solid phase or derivatized to it prior to coating. Examples of solid phases used in immunoassays are porous and non-porous materials, latex particles, magnetic particles, microparticles (see published EPO application No. EP 0 425 633), beads, membranes, microtiter wells and plastic tubes. The choice of solid phase material and method of labeling the antigen or antibody reagent are determined based upon desired assay format performance characteristics. For some immunoassays, no label is required. For example, if the antigen is on a detectable particle such as a red blood cell, reactivity can be established based upon agglutination. Alternatively, an antigen-antibody reaction may result in a visible change (e.g., radial immunodiffusion). In most cases, one of the antibody or antigen reagents used in an immunoassay is attached to a signal generating compound or “label”. This signal generating compound or “label” is in itself detectable or may be reacted with one or more additional compounds to generate a detectable product. Examples of such signal generating compounds include chromogens, radioisotopes (e.g., 125I, 131I, 32P, 3H, 35S, and 14C), fluorescent compounds (e.g., fluorescein, rhodamine), chemiluminescent compounds, particles (visible or fluorescent), nucleic acids, complexing agents, or catalysts such as enzymes (e.g., alkaline phosphatase, acid phosphatase, horseradish peroxidase, beta-galactosidase, and ribonuclease). In the case of enzyme use, addition of chromo-, fluoro-, or lumogenic substrate results in generation of a detectable signal. Other detection systems such as time-resolved fluorescence, internal-reflection fluorescence, amplification (e.g., polymerase chain reaction) and Raman spectroscopy are also useful.

Noncompetitive immunoassays are assays in which antigen is directly detected and, in some instances, the amount of antigen directly measured. Enzyme-mediated immunoassays such as immunofluorescence assays (IFA), enzyme linked immunosorbent assays (ELISA), immunoblotting (western), and capture assays can be readily adapted to accomplish the noncompetitive detection of the MXPyV proteins.

An ELISA method effective for the detection of the MXPyV virus can, for example, be as follows: (1) immobilize an antibody or antigen (e.g. VP1, VP2, VP3, ST-Ag, LT-Ag) to a substrate; (2) contact the immobilized receptor with a fluid or tissue sample containing the virus, a viral antigen, or antibodies to the virus; (3) contact the above with an antibody bound to a detectable moiety (e.g., horseradish peroxidase enzyme or alkaline phosphatase enzyme); (4) contact the above with the substrate for the enzyme; (5) contact the above with a color reagent; (6) observe color change. The above method can be readily modified to detect presence of an anti-MXPyV antibody in the sample or a specific MXPyV protein as well as the virus.

Western blot (immunoblot) analysis can be used to detect and quantify the presence of MXPyV antigen in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the MXPyV antigen. The anti-MXPyV antigen antibodies specifically bind to the MXPyV antigen on the solid support. These antibodies can be directly labeled or alternatively can be subsequently detected using labeled antibodies (e.g., labeled sheep anti-mouse antibodies) that specifically bind to the anti-MXPyV antigen antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. ClM. Prod. Rev. 5:34-41 (1986)).

A MXPyV antigen, e.g. VP1, VP2, VP3, ST-Ag, LT-Ag or epitope-containing portion thereof, and/or a patient's antibodies to the MXPyVvirus can be detected utilizing a capture assay. Briefly, to detect antibodies to MXPyV in a patient sample, antibodies to the patient's immunoglobulin, e.g., anti-IgG (or IgM) are bound to a solid phase substrate and used to capture the patient's immunoglobulin from serum. MXPyV, or reactive fragments of MXPyV, are then contacted with the solid phase followed by addition of a labeled antibody. The amount of patient MXPyV specific antibody can then be quantitated by the amount of labeled antibody binding.

In competitive assays, MXPyV antigen, e.g. VP1, VP2, VP3, ST-Ag, LT-Ag, present in a sample can be detected indirectly by detecting a decrease in a detectable signal associated with a known, added (exogenous) MXPyV antigen displaced (competed away) from an anti-MXPyV antigen antibody by the unknown MXPyV antigen present in a sample.

Competitive assays can also be adapted to provide for an indirect measurement of the amount of MXPyV antigen, e.g. VP1, VP2, VP3, ST-Ag, LT-Ag, present in the sample. Briefly, serum or other body fluids from the subject is reacted with an antibody bound to a substrate (e.g. an ELISA 96-well plate). Excess serum is thoroughly washed away. A labeled (enzyme-linked, fluorescent, radioactive, etc.) monoclonal antibody is then reacted with the previously reacted MXPyV virus-antibody complex. The amount of inhibition of monoclonal antibody binding is measured relative to a control. MABs can also be used for detection directly in samples by IFA for MABs specifically reactive for the antibody-virus complex.

A hapten inhibition assay is another competitive assay. In this type of assay, the known MXPyV antigen, e.g. VP1, VP2, VP3, ST-Ag, LT-Ag, can be immobilized on a solid substrate. The known amount of anti-MXPyV antibody is added to the sample, and the sample is then contacted with the immobilized MXPyV antigen. In a hapten inhibition assay, the amount of anti-MXPyV antibody bound to the known immobilized MXPyV antigen is inversely proportional to the amount of MXPyV antigen present in the sample. The amount of immobilized antibody can be detected by detecting either the immobilized fraction of antibody or the fraction of the antibody that remains in solution. Detection can be direct where the antibody is labeled or indirect by the subsequent addition of a labeled moiety that specifically binds to the antibody as described above.

Immunoassays in the competitive binding format can also be used for crossreactivity determinations. For example, a MXPyV antigen, e.g. VP1, VP2, VP3, ST-Ag, LT-Ag, can be immobilized to a solid support. Proteins can be added to the assay that competes for binding of the antisera to the immobilized antigen. The ability of the added proteins to compete for binding of the antisera to the immobilized protein is compared to the ability of the MXPyV antigen to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera can then be used in a competitive binding immunoassay as described above to compare a second protein, thought to be perhaps an allele or polymorphic variant of a MXPyV antigen, e.g. VP1, VP2, VP3, ST-Ag, LT-Ag, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the MXPyV antigen that is required to inhibit 50% of binding, then the second protein is said to specifically bind to the polyclonal antibodies generated to MXPyV antigen.

Immunoassays (both competitive and non-competitive) also often use a labeling agent to specifically bind to and label the complex formed by the antibody and antigen. The labeling agent can itself be one of the moieties comprising the antibody/antigen complex. Thus, the labeling agent can be a labeled MXPyV protein nucleic acid or a labeled anti-MXPyV antibody. Alternatively, the labeling agent can be a third moiety, such a secondary antibody that specifically binds to the antibody/antigen complex (a secondary antibody is typically specific to antibodies of the species from which the first antibody is derived). Other proteins capable of specifically binding immunoglobulin constant regions, such as protein A or protein G can also be used as a label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art, and can be any material detectable by spectroscopic, photochemical, biochemical, immunochemical, electrical, optical or chemical means. Such detectable labels have been well-developed in the field of immunoassays and can include, but are not limited to, magnetic beads (e.g., DYNABEADS™), fluorescent dyes (e.g., fluorescein isothiocyanate, Texas red, rhodamine, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize MXPyV antigen, or secondary antibodies that recognize anti-MXPyV antigen.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by the use of electronic detectors such as charge coupled devices (CCDs) or photomultipliers and the like. Similarly, enzymatic labels can be detected by providing the appropriate substrates for the enzyme and detecting the resulting reaction product. Colorimetric or chemiluminescent labels can be detected simply by observing the color associated with the label. Thus, in various dipstick assays, conjugated gold often appears pink, while various conjugated beads appear the color of the bead.

Some assay formats do not require the use of labeled components. For instance, a micro-agglutination test can also be used to detect the presence of MXPyV in test samples. Briefly, latex beads are coated with an antibody and mixed with a test sample, such that MXPyV in the tissue or body fluids that is specifically reactive with the antibody crosslink with the receptor, causing agglutination. The agglutinated antibody-virus complexes within a precipitate, visible with the naked eye or by spectrophotometer. Other assays include serologic assays, in which the relative concentrations of IgG and IgM are measured.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

In the diagnostic methods described above, the sample can be taken directly from a subject or in a partially purified form. The antibody specific for a particular MXPyV (the primary reaction) reacts by binding to the virus. Thereafter, a secondary reaction with an antibody bound to, or labeled with, a detectable moiety can be added to enhance the detection of the primary reaction. Generally, in the secondary reaction, an antibody or other ligand which is reactive, either specifically or nonspecifically with a different binding site (epitope) of the virus will be selected for its ability to react with multiple sites on the complex of antibody and virus. Thus, for example, several molecules of the antibody in the secondary reaction can react with each complex formed by the primary reaction, making the primary reaction more detectable.

In other particular embodiments, the methods of the invention involve obtaining a sample from a subject that is suspected of having been exposed to MXPyV or of being infected with MXPyV. Once the requisite sample has been obtained, the sample is contacted with a recombinant MXPyV peptide antigen, e.g. VP1, VP2, VP3, ST-Ag, LT-Ag. Once the sample is contacted with the recombinant MXPyV peptide antigen(s), the presence or absence of antibodies to each of the peptides is then determined in the sample and compared to (1) a predetermined cut-off value, or (2) the intensity of signal generated by one or more controls. In an alternative embodiment, the methods described herein can also be used for detecting MXPyV infection in blood supplies. In still yet a further alternative embodiment, the methods described herein can be used to diagnose MXPyV infection in a subject.

The variety of methods that can be used to monitor specific antibody titer and type in a sample can be classified into two general formats: (1) antigen is immobilized on a solid phase, as described above, the human biological fluid containing the specific antibodies is allowed to react with the antigen, and then antibody bound to antigen is detected with an anti-human antibody coupled to a signal generating compound and (2) an anti-human antibody is bound to the solid phase, the human biological fluid containing specific antibodies is allowed to react with the bound antibody, and then antigen attached to a signal generating compound is added to detect specific antibody present in the fluid sample. In both formats, the anti-human antibody reagent may recognize all antibody classes, or alternatively; be specific for a particular class or subclass of antibody, depending upon the intended purpose of the assay. These assays formats as well as other known formats are intended to be within the scope of the present invention and are well known to those of ordinary skill in the art.

In view of the above, therefore, the present invention includes a method of detecting antibodies to MXPyV in a sample comprising the steps of: (a) contacting the sample suspected of containing the antibodies with a MXPyV antigen or protein, i.e. VP1, VP2, VP3, ST-Ag, LT-Ag, or epitope-containing portion thereof; (b) detecting the presence of the complex and thus antibodies present in the sample. More specifically, the present invention includes a method of detecting antibodies to MXPyV in a sample comprising the steps of: (a) contacting the sample suspected of containing the antibodies with the MXPyV antigen or protein, i.e. VP1, VP2, VP3, ST-Ag, LT-Ag, or epitope-containing portion thereof, for a time and under conditions sufficient to allow the formation of antibody/antigen complexes; (b) adding a conjugate to the resulting antibody/antigen complexes for a time and under conditions sufficient to allow the conjugate to bind to the bound antibody, the conjugate comprising an antibody (directed against the antibodies in the sample) attached to a signal generating compound capable of generating a detectable signal; (c) detecting the presence of the antibody which may be present in the test sample by detecting the signal generated by the signal generating compound. A control or calibrator may also be used which binds to the antigen.

Additionally, the present invention includes another method for detecting the presence of anti-MXPyV antibody which may be present in a sample. This method comprises the steps of: (a) contacting the sample suspected of containing anti-MXPyV antibodies with an anti-antibody specific for the antibody in the sample, e.g. anti-human antibody for a human sample, for a time and under conditions sufficient to allow for formation of anti-antibody/antibody complexes and (b) detecting the presence of antibody which may be present in the test sample. (Such anti-antibodies are commercially available and may be created, for example, by immunizing a mammal with purified mu-chain of the anti-MXPyV antibody raised again the protein of the present invention or epitope-containing portion thereof.)

More specifically, this method may comprise the steps of: (a) contacting the sample suspected of containing the antibodies (i.e., anti-MXPyV antibodies) with anti-antibody specific for the antibodies, under time and conditions sufficient to allow the formation of anti-antibody/antibody complexes; (b) adding a conjugate to the resulting anti-antibody/antibody complexes for a time and under conditions sufficient to allow the conjugate to bind to the bound antibody, the conjugate comprising the protein (i.e., MXPyV antigen or protein, i.e. VP1, VP2, VP3, ST-Ag, LT-Ag, or epitope-containing portion thereof) being attached to a signal generating compound capable of generating a detectable signal; and (c) detecting the, presence of the antibodies which may be present in the test sample by detecting the signal generated by the signal generating compound. A control or calibrator may be used which comprises antibody to the anti-antibody.

The present invention also encompasses a third method for detecting the presence of antibody to MXPyV in a sample. This method comprises the steps of: (a) contacting the sample suspected of containing the anti-MXPyV antibodies with anti-antibody specific for the antibody, under time and conditions sufficient to allow the formation of anti-antibody/antibody complexes; (b) adding protein (i.e., a MXPyV antigen or protein, i.e. VP1, VP2, VP3, ST-Ag, LT-Ag, or epitope-containing portion thereof) to the resulting anti-antibody/antibody complexes for a time and under conditions sufficient to allow the antigen to bind to the antibody; and (c) adding a conjugate to the resulting anti-antibody/antibody/antigen complexes, the conjugate comprising a composition comprising monoclonal or polyclonal antibody attached to a signal generating compound capable of detecting a detectable signal, the monoclonal or polyclonal antibody being directed against the antigen; and (d) detecting the presence of the antibodies which may be present in the test sample by detecting the signal generated by the signal-generating compound. Again, a control or calibrator may be used which comprises antibody to the anti-antibody.

It should also be noted that one or more of the monoclonal antibodies of the present invention may be used as a competitive probe for the detection of antibodies to a MXPyV antigen or protein, i.e. VP1, VP2, VP3, ST-Ag, LT-Ag, or epitope-containing portion thereof. For example, a recombinant MXPyV peptide of the present invention can be coated on a solid phase. A sample suspected of containing antibody to the MXPyV antigen may then be incubated with an indicator reagent comprising a signal-generating compound and at least one monoclonal antibody of the present invention for a time and under conditions sufficient for the formation of antigen/antibody complexes of the test sample and indicator reagent to the solid phase or the indicator reagent to the solid phase. The reduction in binding of the monoclonal antibody to the solid phase can be measured. A measured reduction in the signal as compared to the signal generated from a confirmed negative MXPyV test sample indicates the presence of anti-MXPyV antibody in the test sample.

It should also be noted that the antibodies of the present invention, or fragments thereof, may be utilized in various diagnostic assays in order to determine the presence of MXPyV proteins (or nucleic acid sequences corresponding thereto) in a sample. For example, an antibody directed to one or more of the proteins of the present invention, i.e. MXPyV antigen or protein such as VP1, VP2, VP3, ST-Ag, LT-Ag, or epitope-containing portion thereof, may be added to the sample for a time and under conditions sufficient for the formation of antibody/antigen complexes. If such complexes are detected, then the antigen (i.e., protein) is present in the test sample.

In yet another method, a polyclonal or monoclonal anti-MXPyV antibody or fragment thereof, or a combination of these antibodies, which has been immobilized on a solid phase is contacted with a sample suspected of containing MXPyV proteins, in order to form a first mixture. This mixture is then incubated for a time and under conditions sufficient to form antigen (i.e., protein)/antibody complexes. An indicator reagent comprising a monoclonal or polyclonal antibody, or fragment thereof, which specifically binds to a MXPyV antigen (e.g. VP1, VP2, VP3, ST-Ag, LT-Ag, or epitope-containing portion thereof), or a combination of these antibodies, to which a signal-generating compound has been attached, is then contacted with the antigen/antibody complexes in order to form a second mixture. This second mixture is then incubated for a time and under conditions sufficient for the formation of antibody/antigen/antibody complexes. The presence of a MXPyV protein in the sample and captured on the solid phase is determined by detecting the presence of a measurable signal generated by the signal generating compound. The amount of mutant protein or antigen in the sample is proportional to the signal generated.

Additionally, one may use a different method in order to detect the presence of MXPyV protein in a sample. More specifically, a polyclonal or monoclonal anti-MXPyV antibody (as described above), or a combination thereof, bound to a solid support, the test sample, and an indicator reagent comprising a monoclonal antibody or polyclonal antibody (or fragments thereof) which specifically binds to the MXPyV antigen (e.g., VP1, VP2, VP3, ST-Ag, LT-Ag, or epitope-containing portion thereof), or a combination of these antibodies to which a signal generating compound is attached, are contacted to form a mixture. This mixture is incubated for a time and under conditions sufficient to form antibody/antigen/antibody complexes. MXPyV proteins of the present invention and captured on the solid phase are determined by detecting the measurable signal generated by the signal-generating compound. The amount of MXPyV protein in the test sample is proportional to the signal generated.

It should be noted that one may also detect the presence of antibody and/or antigen to the MXPyV in a simultaneous assay. More specifically, a sample is simultaneously contacted with a capture reagent of a first analyte, which comprises a first binding member specific for the first analyte, attached to a solid phase, and a capture reagent of a second analyte, which comprises a first binding member for a second analyte. (A binding member of a pair is defined as a molecule which, through chemical or physical means, specifically binds to the second molecule of the pair.) A mixture is thus formed. This mixture is then incubated for a time and under conditions sufficient to form capture reagent/first analyte and capture reagent/second analyte complexes. These complexes are then contacted with an indicator reagent comprising a member of a binding pair specific for the first analyte labeled with a signal-generating compound and an indicator reagent comprising a member of a binding pair specific for the second analyte labeled with a signal-generating compound. A second mixture is formed. This second mixture is then incubated for a time and under conditions sufficient to form capture reagent/first analyte/indicator reagent complexes and capture reagent/second analyte/indicator reagent complexes. The presence of one or more analytes is determined by detecting a signal generated in connection with the complexes formed on either or both solid phases as an indication of the presence of one of more analytes in the test sample.

There is a variety of assay formats well-known to those skilled in the art that can be employed using the recombinant MXPyV polypeptides, e.g. VP1, VP2, VP3, ST-Ag, and LT-Ag, described herein to detect antibodies to MXPyV in a sample. For example, in one assay format, one or more of the recombinant peptides can be immobilized on a solid support to bind to and remove one or more antibodies from the test sample. The bound antibody or antibodies can then be detected using a detectable label that binds to the peptide/antibody complex and contains the detectable label. Alternatively, a competitive assay can be utilized, in which an antibody that binds to one or more of the recombinant peptides may be utilized, in which an antibody that binds to one or more of the peptides is labeled with a detectable label and allowed to bind to the immobilized recombinant peptide after incubation with the recombinant peptide in the sample. The extent to which components of the sample inhibit the binding of the labeled antibody to one or more recombinant peptides is indicative of the reactivity of the sample with one or more of the immobilized peptides.

In terms of the detectable label, any detectable label known in the art can be used. For 3 1251 35S_{,} 14C ³²P example, the detectable label can be a radioactive label (such as, e.g., ^{3H}, ^{125I}, ^{38S}, ^{14C}, ^{32P}, and ^{33P}) an enzymatic label (such as, e.g., horseradish peroxidase, alkaline peroxidase, glucose 6-phosphate dehydrogenase, and the like), a chemiluminescent label (such as, e.g., acridinium esters, luminal, isoluminol, thioesters, sulfonamides, phenanthridinium esters, and the like), a fluorescence label (such as, e.g., fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2.sup.nd ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

The solid support can be any material known to those of ordinary skill in the art on which the MXPyV recombinant protein(s), e.g. VP1, VP2, VP3, ST-Ag, LT-Ag, can be immobilized. Examples of solid supports that can be used are a test well in a microtiter plate, nitrocellulose, nylon, a bead or a disc (which can be made out of glass, fiberglass, latex, plastic or a paper material), a gel (for example, a gel through which the polypeptides have been run and which is subsequently dried) or a strip, disc or sheet (which can be made out of nitrocellulose, nylon, plastic or paper). An exemplary assay format for the methods of the present invention is an immunoblot assay in a flow-through or strip test format. In the flow-through or strip test format, the solid support is in the form of a strip, disc or sheet that is made out of nitrocellulose, nylon, plastic or paper. More preferably, the recombinant peptides described herein are immobilized on said strip, disc or sheet. Most preferably, the recombinant proteins are arranged as separate, parallel bands, spots or dots on the strip, disc or sheet (each of which may be referred to as a “test” band, spot or dot, collectively as “test” bands, spots or dots). The recombinant proteins can be immobilized on said strip, disc or sheet using routine techniques known in the art, such as automated techniques, such as by jetting the recombinant proteins on to said strip, disc or sheet (using a jetting instrument such as those available from Bio-Dot ((such as the AJQ3000 Air Jet Quanti or the RR 4200-Dip Tank), Irvine, Calif.) or manual techniques, such as by pipetting the recombinant proteins on to said strip, disc or sheet. If a sheet is used, once all of the recombinant peptides are immobilized onto the sheet, the sheet can be cut, using routine techniques known in the art into strips for use in an assay. The location of the recombinant peptides (and optionally, any controls) on the strip, disc or sheet is not critical. Additionally, the strip, disc or sheet can be further immobilized on a support layer using routine techniques known in the art, such as gluing, lamination, etc. The support layer can be made from plastic, cardboard, etc. For example, a nitrocellulose strip or disc can be laminated onto a pressure-sensitive plastic film. Further optionally, in addition to any discrete region employed for the location of an on-board control or test bands, spots or dots, the strip disc or sheet optionally comprises an identification region employed for labeling a sample such that it can be differentiated from other samples (e.g., name, number, alphanumeric reference, bar code, or other appropriate means).

The recombinant peptides (namely, e.g. VP1, VP2, VP3, ST-Ag, LT-Ag) may be bound to or immobilized on to the solid support using any techniques known to those skilled in the art (for example, using a Western blot technique, the method for which is well known to those skilled in the art). In addition, and optionally, one or more controls can also be immobilized on to the solid support (such as for use in an immunoblot assay, namely, in a flow-through or strip test format). The terms “bound” or “immobilized” as used interchangeably herein, refer to both the noncovalent association, such as adsorption, and covalent attachment (which may be a direct linkage between the recombinant protein) and the functional groups on the solid support or may be a linkage that is effected by way of a cross-linking agent). Binding by adsorption to a strip, disc or sheet is preferred. In such instances, adsorption can be achieved by contacting solutions of each of the recombinant peptides, and optionally, any control in a suitable buffer, with the strip, disc or sheet for a suitable amount of time. The contact time will vary depending on the temperature, but is between about 1 hour and about 24 hours.

If necessary, covalent attachment of the recombinant peptides (and optionally, any controls) to a solid support can be achieved by first reacting the solid support with a bifunctional reagent that will react with both the support and a functional group, such as a hydroxyl or amino group, on the recombinant peptides. For example, the peptides may be bound to supports having an appropriate polymer coating using benzoquinone or by condensation of an aldehyde group on the support with an amine and an active hydrogen on the peptide.

Once the recombinant peptides (and optionally, any controls) are immobilized on the support, the remaining protein binding sites on the support are typically blocked. Any suitable blocking agent known to those of ordinary skill in the art can be used. For example, bovine serum albumin (“BSA”), phosphate buffered saline (“PBS”) solutions of casein in PBS, Tween 20™ (Sigma Chemical Company, St. Louis, Mo.), as well as other blocking agents, can be employed. Optionally, for use of a support comprising a gel which is subsequently dried, blocking of the support may not be necessary. After blocking is completed, the support can optionally be washed, such as with PBS and allowed to dry (such as by air drying) for a suitable amount of time. The drying time will vary depending on the temperature, but is between about 30 minutes and about 24 hours.

The immobilized recombinant peptides (and optionally, one or more controls) are then allowed to incubate with the test sample. Prior to said incubation, the test sample may be diluted with a suitable diluent, such as PBS. During this incubation, if any antibodies are present in the test sample, these antibodies will bind to one or more of the recombinant peptides on the solid support. Generally, the incubation period is a period of time that is sufficient to permit the detection of the presence of MXPyV antibodies within the sample. Preferably, the incubation period is between about 15 minutes to about 6 hours. Most preferably, the incubation period is between about 1 hour and about 4 hours.

Unbound test sample may be removed by washing the solid support with an appropriate buffer, such as PBS or a Tris buffer (such as a Tris buffer containing 20 mM Tris, 0.15% Tween 20™ and 0.1% sodium azide). One or more detectable reagents can be added to the solid support. Appropriate detectable reagents are any compounds that binds to the immobilized peptide-antibody complex (and optionally any immobilized controls) and that can be detected by any of a variety of means that are known to those skilled in the art. Preferably, the detectable reagent contains a binding agent, such as, for example, Protein A, Protein G, an immunoglobulin, a lectin or a free antigen) conjugated to a detectable label. The conjugation of the binding agent to the detectable label can be achieved using standard methods known to those skilled in the art. Common binding agents may be purchased conjugated to a variety of detectable labels from a number of commercial sources, including, but not limited to Zymed Laboratories (San Francisco, Calif.) and Pierce (Rockford, Ill.).

One or more detection reagents are incubated with the immobilized peptide-antibody complex (and optionally, one or more controls) for an amount of time that is sufficient to detect the bound antibody or antibodies (and optionally, one or more controls). A suitable incubation time can generally be determined from the manufacturer's instructions or by assaying the level of binding that occurs over a period of time. Unbound detection reagent can then be removed and bound detection reagent is detected using the detectable label. The method used for detecting the detectable labels will depend on the nature of the detectable labels used in the assay. For example, for radioactive labels, scintillation counting or autoradiographic methods can be used. For chemiluminescent or fluorescent labels, spectroscopic methods can be used. Enzymatic labels can generally be detected by the addition of a substrate (usually for a specific period of time), followed by spectroscopic or other analysis of the reaction products.

To determine the presence or absence of antibodies to MXPyV in the test sample, the signal(s) detected from the detectable label(s) that remain bound to the solid support is compared to a pre-determined cut-off value. More specifically, this cut-off value can be the average mean signal obtained when the immobilized recombinant proteins are incubated with samples from a subject that is not infected with MXPyV. In general, a sample generating a signal that is three standard deviations above the mean is considered positive for MXPyV antibodies and MXPyV infection. Alternatively, if a light, darkness or color reading apparatus, such as a densitometer, that is capable of generating a numerical value is employed, the cut-off value can be determined using a Receive Operator Curve (“ROC”), using the method of Sackett et al., Clinical Epidemiology: A Basic Science for Clinical Medicine, p. 106-107 (Little Brown and Co., 1985). Briefly, the cut-off value may be determined from a plot of pairs of true positive rates (namely, sensitivity) and false positive rates (namely, 100% specificity) that correspond to each possible cut-off value for the diagnostic test result. The cut-off value on the plot that is the closest to the upper left-hand corner (namely, the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate. In general, a sample generating a signal that is higher than the cut-off value determined by this method is considered positive for MXPyV infection. In some embodiments, the identification of a signal demonstrating the binding of antibodies to at least 1, at least 2, at least 3, or at least 4 of the 5 recombinant proteins VP1, VP2, VP3, ST-Ag, and LT-Ag indicates the presence of MXPyV in said sample.

As discussed previously herein, the preferred assay format is an immunoblot assay, e.g. a flow-through or strip format, wherein one or more recombinant protein(s) selected from VP1, VP2, VP3, ST-Ag, and LT-Ag are immobilized on a strip, disc or sheet, such as a nitrocellulose, nylon, plastic or paper strip, disc or sheet, e.g. as separate test bands, spots or dots. The immobilization of each of these recombinant peptides on a strip, disc or sheet can be obtained using the techniques described previously herein. Moreover, in some embodiments, in addition to the above recited recombinant peptides, the strip, disc or sheet also contains immobilized thereon as separate test bands, spots or dots at least one control (each of which may be referred to as an “on-board control” band, spot or dot, collectively as “on-board control” bands, spots or dots). It is preferred that the strip, disc or sheet contain immobilized thereon two separate, discrete controls (namely, a first control and a second control), most preferably, the strip, disc or sheet can contain immobilized thereon three separate, discrete controls (namely, a first control, a second control and a third control). If more than one control is present, then the controls may be identical to one another or different from one another. Preferably, at least two of the controls are identical (such as, for example, the first control and the second control). If two of the controls are identical, it is preferred that the concentration of one of the controls (either the first control or the second control or if three controls are present, the first control or the third control or the second control or third control) immobilized on the strip, disc or sheet be higher (or greater) than the other control immobilized on the strip, disc or sheet. The control immobilized on the strip, disc or sheet in a higher concentration than the other control is referred to as the “high control”. The control immobilized on the strip, disc or sheet in a lower concentration than the high control is referred to as the “low control”. The ratio of the concentration of low control to high control present on the strip, disc or sheet can be from about 1:2 to about 1:10, preferably, about 1:5 to about 1:6. For example, the first control may be the low control and the second control may be the high control. Alternatively, the first control may be the high control and the second control may be the low control. By way of another example, the strip, disc or strip can contain 3 controls, namely, a low control and a high control as well a third control (which can be used, for example, to verify sample addition). The low control and high control can both be human IgG (wherein the ratio of low control to high control is from about 1:2 to about 1:10) and the third control can be a goat anti-human IgG.

In the flow-through format, one end of the strip, disc or sheet at which the recombinant peptides are bound can be immersed in a solution containing the sample. Alternatively, the entire strip, disc or sheet can be placed in a reaction tray along with a diluent and then the sample added to the reaction tray. The sample and strip are allowed to incubate for a sufficient period of time using the same times and techniques described previously herein. Unbound sample components can be removed using the techniques described previously herein. In this format, antibodies within the sample bind to the immobilized peptides (and the at least one control) as the test sample passes through the membrane. At least one detection reagent (such as a detection reagent described previously herein containing a detectable label) can be added. The at least one detection reagent binds to each of the peptides and peptide-antibody complexes formed as the solution containing the detection reagent flows through the strip. To determine the presence or absence of MXPyV antibodies in the test sample, the detection of the bound detection reagents can be performed as described above using the a cut-off or by comparing the intensity of one or more signals generated by one or more controls as discussed in more detail below.

When a low control and high control as described above are used in the flow-through format, it is preferred that the presence or absence of the MXPyV antibodies in the sample be determined by identifying the presence of a signal from the detectable label at each of the test bands (or spots or dots) for the peptides. If a signal is identified at a test band for a peptide, then the intensity of this detected signal is compared with the intensity of the signal from the low control band (or spot or dot) and the high control band (or spot or dot), using a scale of 0 to 4+. The reading is 0 when no band is visible. The intensities of the low control band and high control band are defined as 1+ (for the low control) and 3+ (for the high control), respectively. A test band with an intensity comparable to that of the low control would be rated 1+. A band with intensity between that of the low control and the high control band would be rated 2+. A band with an intensity comparable to that of the high control would be rated 3+. A band intensity higher than that of the high control would be rated 4+. A faint band with intensity weaker than that of the low control would be rated .+−. If the results of the immunoblot assay are that no bands are visible (other than the bands for the low control, high control and negative control), then the test sample is deemed to be negative for MXPyV. If the results of the immunoblot assay are that one or more bands for the recombinant polypeptides demonstrate a signal, then the following analysis must be performed. Specifically, if all of the bands exhibit an intensity that is weaker than the low control, namely, all of the bands are rated .+−., then the test sample is deemed to be indeterminate for MXPyV. However, if at least one of the bands is rated 1+ or higher, then the test sample is considered to be positive for MXPyV antibodies.

While the present invention discloses the use of solid phase diagnostic assays, it is contemplated that the proteins of the present invention may be utilized in non-solid phase diagnostic assays. These assays are well-known to those of ordinary skill in the art and are considered to be within the scope of the present invention.

### Assays for Immune Modulators for MXPyV

Immune modulators for MXPyV can be discovered using a variety of in vitro and in vivo assays, including cell-based models. Compound candidates are tested using either a recombinant or naturally occurring protein of choice. Modulation can include, but is not limited to, modulation of infection, replication, receptor binding, cell entry, particle formation, and the like.

Measurement of modulation of a MXPyV or a cell expressing MXPyV, either recombinant or naturally occurring, can be performed using a variety of assays, in vitro, in vivo, and ex vivo, as described herein. A suitable physical, chemical or phenotypic change that affects activity, e.g., enzymatic activity, cell surface marker expression, viral replication and proliferation can be used to assess the influence of a test compound on the polypeptide of this invention. When the functional effects are determined using intact cells or animals, one can also measure a variety of effects.

Assays to identify compounds with MXPyV modulating activity can be performed in vitro. Such assays can use full length MXPyV or a variant thereof, or a mutant thereof, or a fragment thereof. Purified recombinant or naturally occurring protein can be used in the in vitro methods of the invention. In addition to purified MXPyV, the recombinant or naturally occurring protein can be part of a cellular lysate or a cell membrane. As described below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

A high throughput binding assay can be performed in which the protein or a fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described below, including small organic molecules, peptides, antibodies, etc. A wide variety of assays can be used to identify MXPyV-modulator binding, including labeled protein-protein binding assays, electrophoretic mobility shifts, immunoassays, enzymatic assays, and the like. In some cases, the binding of the candidate modulator is determined through the use of competitive binding assays, where interference with binding of a known ligand or substrate is measured in the presence of a potential modulator. Either the modulator, the known ligand, or substrate is bound first; then the competitor is added. After the protein is washed, interference with binding, either of the potential modulator or of the known ligand or substrate, is determined Often, either the potential modulator or the known ligand or substrate is labeled.

A cell-based assay can be used in which the MXPyV is expressed in a cell, and functional, physical, chemical and phenotypic changes are assayed to identify viral modulators. Any suitable functional effect can be measured as described herein, in addition to viral inhibition assays as are well known in the art. The MXPyV can be naturally occurring or recombinant. Also, fragments of the MXPyV or chimeric proteins thereof can be used in cell based assays. In addition, point mutants in essential residues required by the catalytic site can be used in these assays.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., I Amer. Chem. Soc. 116:2661 (1994)), oligocarbamates (Cho et al., Science 261:1303 (1993)), and/or peptidyl phosphonates (Campbell et al., I Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

A solid state or soluble high throughput assay can be performed using MXPyV, or a cell or tissue infected with MXPyV (either naturally occurring or recombinant). A solid phase in vitro assay can be used in a high throughput format where MXPyV is attached to a solid phase. Any one of the assays described herein can be adapted for high throughput screening.

In high throughput assays, either soluble or solid state, it is possible to screen up to several thousand different modulators or ligands in a single day. This methodology can be used for MXPyV in vitro, or for cell-based or membrane-based assays comprising a MXPyV. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage. A tag for covalent or non-covalent binding can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like (see, e.g., Pigott & Power, The Adhesion Molecule Facts Book I (1993)). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates)). Non-chemical approaches for fixing tag binders to substrates include other common methods, such as heat, cross-linking by UV radiation, and the like.

The compounds tested as modulators of MXPyV can be any small organic molecule, or a biological entity, such as a protein, e.g., an antibody or peptide, a sugar, a nucleic acid, e.g., an antisense oligonucleotide or a ribozyme or siRNA, or a lipid. Alternatively, modulators can be genetically altered versions of a MXPyV. Typically, test compounds will be small organic molecules, peptides, circular peptides, siRNA, antisense molecules, ribozymes, and lipids.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

### Treating/Preventing MXPyV

Embodiments described herein further relate to the therapeutic, prophylactic and research uses of various techniques to block or modulate the expression of MXPyV viral proteins or propagation of the virus. Modulators of MXPyV useful for treating or preventing MXPyV can include, but is not limited to MXPyV antigens, genetically modified versions of MXPyV, e.g., versions with altered activity, as well as naturally occurring and synthetic ligands, substrates, antagonists, agonists, antibodies, peptides, cyclic peptides, aptamers, nucleic acids, antisense molecules, ribozymes, siRNA molecules, miRNA molecules, and small chemical molecules, as is well known in the art.

Further described herein are immunogenic compositions for therapeutic or prophylactic purposes against MXPyV. Within certain aspects, MXPyV virus, proteins or peptides and immunogenic fragments thereof, and/or polynucleotides, as well as anti-MXPyV antibodies and/or T cells, can be incorporated into pharmaceutical compositions or immunogenic compositions. Whole virus vaccines (live and attenuated, or replication incompetent, or killed) or subunit vaccines, such as structural or non-structural MXPyV proteins or immunogenic fragments thereof, can be used to treat or prevent MXPyV infections by eliciting an immune response in a subject. Alternatively, a pharmaceutical composition can comprise an antigen-presenting cell (e.g., a dendritic cell) transfected with a MXPyV polynucleotide such that the antigen-presenting cell expresses a MXPyV peptide.

Nucleic acid vaccines encoding a genome, structural protein or non-structural protein or a fragment thereof of MXPyV can also be used to elicit an immune response to treat or prevent MXPyV infection. Numerous gene delivery techniques are well known in the art, such as those described by Rolland (1998) Crit. Rev. Therap. Drug Carrier Systems 15:143-198, and references cited therein. Appropriate nucleic acid expression systems contain the necessary DNA sequences for expression in the patient (such as a suitable promoter and terminating signal). In a preferred embodiment, the DNA can be introduced using a viral expression system (e.g., vaccinia, pox virus, retrovirus, or adenovirus), which can involve the use of a non-pathogenic (defective), replication competent virus. Suitable systems are disclosed, for example, in Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, 4,777,127 and 5,017,487; WO 89/01973; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; and Guzman et al. (1993) Cir. Res. 73:1202-1207. Techniques for incorporating DNA into such expression systems are well known to those of ordinary skill in the art. The DNA can also be “naked,” as described, for example, in Ulmer et al. (1993) Science 259:1745-1749 and reviewed by Cohen (1993) Science 259:1691-1692. The uptake of naked DNA can be increased by coating the DNA onto biodegradable beads, which are efficiently transported into the cells. It will be apparent that a vaccine can comprise both a polynucleotide and a polypeptide component. Such vaccines can provide for an enhanced immune response.

Vaccine preparation is generally described in, for example, Powell and Newman, eds., Vaccine Design (the subunit and adjuvant approach), Plenum Press (NY, 1995). Vaccines can be designed to generate antibody immunity and/or cellular immunity such as that arising from CTL or CD4+ T cells.

A non-specific immune response enhancer can be any substance that enhances an immune response to an exogenous antigen. Examples of non-specific immune response enhancers include adjuvants, biodegradable microspheres (e.g., polylactic galactide) and liposomes (into which the compound is incorporated; see, e.g., U.S. Pat. No. 4,235,877). Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, can also be used as adjuvants.

### Pharmaceutical Compositions

Pharmaceutical compositions and vaccines within the scope of the present invention can also contain other compounds, which can be biologically active or inactive. For example, one or more immunogenic portions of other antigens can be present, either incorporated into a fusion polypeptide or as a separate compound, within the composition or vaccine. Polypeptides can, but need not be, conjugated to other macromolecules as described, for example, within U.S. Pat. Nos. 4,372,945 and 4,474,757. Pharmaceutical compositions and vaccines can generally be used for prophylactic and therapeutic purposes.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention can be formulated as a lyophilizate. Compounds can also be encapsulated within liposomes using well known technology.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to affect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

Pharmaceutical and vaccine compositions can be presented in unit-dose or multi-dose containers, such as sealed ampoules or vials. Such containers are preferably hermetically sealed to preserve sterility of the formulation until use. In general, formulations can be stored as suspensions, solutions or emulsions in oily or aqueous vehicles. Alternatively, a vaccine or pharmaceutical composition can be stored in a freeze-dried condition requiring only the addition of a sterile liquid carrier immediately prior to use.

### Kits

The invention further provides diagnostic reagents and kits comprising one or more such reagents for use in a variety of diagnostic assays, including for example, immunoassays such as ELISA and “sandwich” type immunoassays, as well as nucleic acid assay, e.g., PCR assays. In a related embodiment, the assay is performed in a flow-through or strip test format, wherein the binding agent is immobilized on a membrane, such as nitrocellulose. Such kits can preferably include at least a first peptide, or a first antibody or antigen binding fragment of the invention, a functional fragment thereof, or a cocktail thereof, or a first oligo pair, and means for signal generation. The kit's components can be pre-attached to a solid support, or can be applied to the surface of a solid support when the kit is used. The signal generating means can come pre-associated with an antibody or nucleic acid of the invention or can require combination with one or more components, e.g., buffers, nucleic acids, antibody-enzyme conjugates, enzyme substrates, or the like, prior to use.

Kits can also include additional reagents, e.g., blocking reagents for reducing nonspecific binding to the solid phase surface, washing reagents, enzyme substrates, enzymes, and the like. The solid phase surface can be in the form of microtiter plates, microspheres, or other materials suitable for immobilizing nucleic acids, proteins, peptides, or polypeptides. An enzyme that catalyzes the formation of a chemiluminescent or chromogenic product or the reduction of a chemiluminescent or chromogenic substrate is one such component of the signal generating means. Such enzymes are well known in the art. Where a radiolabel, chromogenic, fluorigenic, or other type of detectable label or detecting means is included within the kit, the labeling agent can be provided either in the same container as the diagnostic or therapeutic composition itself, or can alternatively be placed in a second distinct container means into which this second composition can be placed and suitably aliquoted. Alternatively, the detection reagent and the label can be prepared in a single container means, and in most cases, the kit will also typically include a means for containing the vial(s) in close confinement for commercial sale and/or convenient packaging and delivery.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

### Identification of a New Human Polyomavirus (MXPyV)

### Methods

### Stool Sample Collection, Nucleic Acid Extraction, and Illumina Deep Sequencing

Anonymized samples were collected from 96 children with acute diarrheal disease from 3 different states in Mexico between 2008-2009. Diarrhea was defined as three or more loose or liquid stools per day, and samples were taken from children prior to treatment with rehydration and antibiotics (if indicated). Viral particles were purified from stool samples by generating a suspension consisting of 1 mL phosphate-buffered saline, 0.1 g of glass beads, 100 μL of chloroform, and 0.2 g of feces, shaking×5 min using a mechanical shaker, spinning×20 min at 1,000 g in a centrifuge, and recovering the supernatant. 500 μL of supernatant were then passed through a 0.45 μm filter and treated with a cocktail of nucleases (Turbo DNAse, Ambion and RNAseA, Invitrogen) prior to nucleic acid extraction using the PureLink 96 Viral RNA/DNA Kit (Invitrogen). Sample cDNA libraries were prepared from extracted nucleic acid using a random PCR amplification method, separately barcoded, and sequenced on an Illumina HiSeq 2000 as previously described (10, 28). Raw Illumina sequences consisting of 75 base pair (bp) paired-end reads were filtered to exclude low-complexity, homopolymeric, and low-quality sequences, and then processed through an automated pipeline for pathogen identification as previously described (28). Sequences corresponding to MXPyV were identified on the basis of viral BlastX homology at a threshold E-score cutoff of 10⁻⁵.

### PCR for Genome Recovery

Three contigs (contiguous sequences) were assembled from deep sequencing reads bearing homology to polyomaviruses by viral BlastX alignment (marked “C1”, “C2”, and “C3” in FIG. 1). To bridge these contigs, long-range PCR was performed using primers directed outward from the assembled contigs and the PrimeStar GXL DNA Polymerase kit (Takara Bio) according to the manufacturer's instructions. Overlapping PCR products were cloned and sequenced in order to obtain a consensus sequence for the complete MXPyV genome at 3× redundancy. Open reading frames were identified using Geneious software (19).

### Phylogenetic Analysis

Whole-genome sequences corresponding to all known animal and human polyomaviruses, with the exception of the recently discovered MWPyV and HPyV10 viruses, were downloaded from GenBank. Multiple sequence alignments of MXPyV viral proteins relative to corresponding proteins from other polyomaviruses were performed using MAFFT (v6.0) with the E-INS-i option and at default settings (34). Overall pairwise amino acid identities of MXPyV relative to other polyomaviruses were calculated by concatenating the VP1, VP2, and T-antigen protein sequences and running MAFFT. To generate the phylogenetic trees, Bayesian tree topologies were calculated using MrBayes V3.2 software (5,500 sampled trees; 500 trees discarded as burn-in for VP1, VP2, and small T antigen; 20,000 sampled trees; 10,000 trees discarded as burn-in for large T antigen needed to achieve convergence) (48). Bovine polyomavirus (FIG. 2, “Bovine”) was selected as an outgroup. Convergence was confirmed by the PSRF statistic in MrBayes. Trees were visualized using Geneious software (19). Multiple whole-genome sequence alignments of MXPyV, HPyV10, and MWPyV were performed using Geneious software (19).

### PCR-Based Screening for MXPyV

A real-time quantitative RT-PCR (qRT-PCR) assay was designed for detection of MXPyV from the VP1 gene, as were two secondary conventional RT-PCR assays from another region of the VP1 gene and the large T-antigen. A reverse transcription step was included for all of the assays in order to enable detection of MXPyV viral mRNA in addition to genomic DNA. To investigate the relative contribution of MXPyV mRNA to viral detection and assess titers of genomic MXPyV, we also performed real-time qPCR on samples found to be MXPyV-positive by qRT-PCR. A standard curve was calculated from 3 PCR replicates at 8 serial log dilutions of a quantified 137-bp MXPyV PCR amplicon. Assays were performed with the Qiagen One-Step RT-PCR kit using 13.5 μL H₂O, 5 μL 5× buffer, 1 μL dNTP, 1 μL RT/Taq mix, 1.5 μL of forward and reverse 10 μM primers, 0.5 μL of 2.5× SybrGreen (for the real-time assay), and 2 μL of extracted nucleic acid. MXPyV primers for the PCR assays are listed in Supplementary Table 2. A sample was considered positive for MXPyV if confirmed by Sanger sequencing or if at least two of the three RT-PCR assays was also positive.

### Prevalence Study Populations

Mexico.

Stool samples from 96 children with diarrheal disease (including the initial MXPyV-positive case identified) were extracted and tested for MXPyV by PCR. Nasal washes from 136 hospitalized children with pneumonia collected from 2010-2012 were extracted using the PureLink 96 Viral RNA/DNA Kit (Invitrogen) and tested for MXPyV.

California (SIFT Study).

The stool samples corresponding to the SIFT (Stanford Infection and Familial Transmission) study have been described previously [38]. Briefly, 553 stool samples from 406 individuals, nearly all children, with or without symptoms of gastroenteritis, were available for study. Stool samples were collected around the time of an initial gastroenteritis episode, and individuals were surveyed for the presence or absence of diarrhea, vomiting, or both within the prior 2 weeks. Additional stool samples were also occasionally collected 3 months after the initial episode. Stool was suspended in 2 mL of PBS at 10% weight per volume and the PureLink 96 Viral RNA/DNA Kit (Invitrogen) was used to extract nucleic acid for MXPyV testing.

Chile.

192 samples (96 from children with diarrhea and 96 from age-/sex-matched controls) collected between 2009-2011 from Chile were available for testing. Viral particles were enriched by filtration and nuclease treatment prior to nucleic acid extraction using the QIAAMP Viral Ultrasens Kit (Qiagen).

California (UCSF Study).

193 plasma samples from solid organ and bone marrow transplant recipients at UCSF sent in 2012 for cytomegalovirus (CMV) testing, with 31 (16%) samples positive for CMV, and 287 plasma/urine samples from predominantly renal transplant recipients sent in 2012 for BKV testing, with 162 (56%) samples positive for BKV, were tested for MXPyV. Viral DNA extractions were performed using the automated Qiagen EZ1 instrument (Qiagen) according to the manufacturer's protocol.

Nucleotide Sequence Accession Numbers

The annotated, complete genome of MXPyV has been submitted to GenBank (accession number JX259273). Deep sequencing reads corresponding to the diarrheal stool library from which MXPyV was identified have been submitted to the NCBI Sequence Read Archive (accession number SRA056896). All ViroChip microarrays used in this study have been deposited in the NCBI GEO database (accession numbers GSE40008; GSM983236-GSM983247). Accession numbers for the animal and human polyomaviruses used in the phylogenetic analysis are listed as follows: NC_{—}015150, NC_{—}014743, NC_{—}014407, NC_{—}014406, NC_{—}014361, NC_{—}013796, NC_{—}013439, NC_{—}012122, NC_{—}011310, NC_{—}010277, NC_{—}009951, NC_{—}009539, NC_{—}009238, NC_{—}007923, NC_{—}007922, NC_{—}004800, NC_{—}004764, NC_{—}004763, NC_{—}001699, NC_{—}001669, NC_{—}001663, NC_{—}001538, NC_{—}001515, NC_{—}001505, and NC_{—}001442.

SUPPLEMENTARY TABLE 2
PCR primer sequences used for MXPyV whole-genome assembly, MXPyV
screening, and diarrheal virus screening.

| | |
|---|---|
| Primer | Sequence |

Primers for MXPyV Whole-Genome Assembly

| | | |
|---|---|---|
| MX-VP1-77F | GGTTGAAGAATGACCTCAACTGTC | SEQ ID NO: 8 |
| MX-VP1-231R | GTATATGTGGGAGGCAGTTGTTC | SEQ ID NO: 9 |
| MX-VP3-F | GACAGACTCCTGATTGGATG | SEQ ID NO: 10 |
| MX-largeT-F | CCTGTAGATTTTCCTGAAGTACTC | SEQ ID NO: 11 |
| MX-VP1-1F | CGGACACCACAATGACAGTTGA | SEQ ID NO: 12 |
| MX-VP3-R | GCTTCTGCTCTGGTACAAACAG | SEQ ID NO: 13 |
| MX-largeT-R1 | GCTCACTGTACACAGATTTGAAC | SEQ ID NO: 14 |
| MX-2500-1F | AAGCTACAGACTGGGTCAC | SEQ ID NO: 15 |
| MX-2500-2R | TCTTCTCTGAGCAGTGAC | SEQ ID NO: 16 |
| MX-2500-1R | CTACAGTATTACTGGATG | SEQ ID NO: 17 |
| MX-2500-2F | CTGCTGTTACATATAGCC | SEQ ID NO: 18 |
| MX-2000-2R | GTCCAGAGTTAACCTGTG | SEQ ID NO: 19 |
| MX-2000-3R | CCTGGATATAGACACTTTG | SEQ ID NO: 20 |
| MX-2500-4004 | GTGTCGTCACTTGGCATA | SEQ ID NO: 21 |
| MX-2500-3607 | ATAGTAATAATACCTGGG | SEQ ID NO: 22 |
| MX-smallT- | TTAAAACTGCACCCTGAC | SEQ ID NO: 23 |
| 1724 | | |

Primers for MXPyV screening

| | | |
|---|---|---|
| MX-Scr-VP1- | GAGGCCTGGGCTCCAGATC | SEQ ID NO: 24 |
| 523F | | |
| MX-Scr-VP1- | CCCACACCTCTATCATCCAG | SEQ ID NO: 25 |
| 660R | | |
| MX-Conf- | CCTGTAGATTTTCCTGAAGTACTC | SEQ ID NO: 26 |
| largeT-1F | | |
| MX-Conf- | GCTCACTGTACACAGATTTGAAC | SEQ ID NO: 27 |
| LargeT-2R | | |
| MX-Conf- | CCCYTGTGTAARGGAGATGGG | SEQ ID NO: 28 |
| VP1-661Fdeg | | |
| MX-Conf- | AGGRTAAGGATTTYTAACAGCYCTT | SEQ ID NO: 29 |
| VP1-807Rdeg | | |

Primers for Diarrheal Virus Screening

| Primer | Sequence | Reference | PMID |
|---|---|---|---|
| Calici-F | GATTACTCCARGTGGGAYTCMAC | Farkas et al. | 15221533 |
| | SEQ ID NO: 30 | (2004) | |
| Calici-R | TGACRATKTMATCATCMCCRTA | | |
| | SEQ ID NO: 31 | | |
| Adeno-F | GCCGCAGTGGTCTTACATGCACATC | Echavarria et al. | 9774586 |
| | SEQ ID NO: 32 | (1998) | |
| Adeno-R | CAGCACGCCGCGGATGTCAAAGT | | |
| | SEQ ID NO: 33 | | |
| Entero-F | CGGCCCCTGAATGCGGCTAA | Rotbart et al. | 2157735 |
| | SEQ ID NO: 34 | (1990) | |
| Entero-R | ATTGTCACCATAAGCAGCC | Benschop et al. | 16355330 |
| | SEQ ID NO: 35 | (2006) | |
| Rota-F | AAGTAGCTGGATTTGATTATTC | Schwarz et al. | 12270660 |
| | SEQ ID NO: 36 | (2002) | |

Pan-Viral Microarray (ViroChip) Analysis of MXPyV-Positive Samples from Mexico

Sufficient material was available from the stool samples from Mexico to test the 12 MXPyV-positive samples for co-infections by pan-viral microarray (ViroChip) and specific PCR analysis for diarrheal viruses. ViroChip analysis was performed as previously described (10, 28). Briefly, RNA was reverse-transcribed to cDNA using random primers (5′-GTTCCCACTGGAGGATA(N₉)-3′) (SEQ ID NO: 37) and second-strand synthesis was performed using Sequenase. Samples were labeled with Cy3 fluorescent dye, normalized to 10 pmol of incorporated dye, and hybridized overnight to the ViroChip microarray for 16 hr at 65° C. The current 8×60 k version 5.0 (v5.0) ViroChip microarrays used in this study (GEO accession number GPL15905) are manufactured commercially on an Agilent platform (Agilent Technologies), and contain 19,058 70mer oligonucleotide probes representing all viral species in GenBank. Microarrays were scanned at 2 μm resolution on an Agilent DNA Microarray Scanner. Microarray hybridization patterns were interpreted using cluster and single oligonucleotide Z-score analysis as previously described (10, 15, 22, 28). Samples were declared positive for a diarrheal virus by microarray if positive by both cluster and Z-score analysis.

Diarrheal Viral PCR Analysis of MXPyV-Positive Samples from Mexico

PCR for 5 diarrheal viruses (calicivirus, astrovirus, adenovirus, rotavirus, and enterovirus) was performed using randomly amplified cDNA as a template. Primer pairs are listed in Supplementary Table 2. All PCR assays were run in a total of 20 μL with 1×PCR buffer, 2 mM MgCl₂, 0.3 mM dNTP, 10 pmol of each primer, and 1 unit of Taq DNA Polymerase (Invitrogen). Calicivirus, rotavirus, and enterovirus PCRs were run at 94° C.×2 min; 35 cycles of 94° C. for 30 s, 50° C. for 30 s, 72° C. for 1 min; and extension at 72° C. for 5 min. Adenovirus and astrovirus PCRs were run at 94° C.×2 min; 35 cycles of 94° C. for 30 s, 55° C. for 30 s, 72° C. for 1 min; and final extension at 72° C.×5 min. Products were visualized on a 1.5% agarose gel stained with ethidium bromide.

### Results

### Discovery and Whole-Genome Sequencing of MXPyV

Eighty stool samples selected from an ongoing investigation of pediatric gastroenteritis from Mexico were analyzed by unbiased Illumina paired-end sequencing. Samples were individually barcoded and sequenced in pools containing 16 samples each. Each pool was subjected to an automated viral discovery pipeline by GenBank database searches and categorization into human, bacterial, phage, unknown, and viral sequences (28). In one pool consisting of 79,013,460 paired-end sequences, three 100-bp reads, all derived from a single barcoded sample from a 2-year child with diarrhea, were found to have amino acid homology to polyomaviruses by BLASTx. These 3 reads and their corresponding mate pairs were aligned using BLASTn at a E-score cutoff of 10⁻¹⁰ to the full deep sequencing dataset corresponding to the barcoded sample (17,981,772 reads) and the resulting identified read pairs assembled to generate 3 contigs (contiguous sequences) 192, 275, and 261 bp in length (FIG. 1, “C1”, “C2”, and “C3”). The closest protein hits to the translated C1, C2, or C3 contigs in the GenBank viral database included VP3 from orangutan polyomavirus (GenBank CAX87756, E-score=9×10⁻¹¹, 81% identity), VP1 from TSV (GenBank YP_{—}003800006, E-score=7×10⁻³⁰, 52% identity), and the large T antigen from orangutan polyomavirus (GenBank CAX87759, E-score=1×10⁻²⁵, 61% identity), respectively. Using long-range PCR with primers directed outward from each of the 3 contigs, the entire genome of the novel polyomavirus was then cloned and sequenced from three overlapping fragments by long-range PCR.

### Genomic Organization and Phylogenetic Analysis

The genome of MXPyV is circular and 4,939 nt in length (accession number JX259273), encoding predicted full-length open reading frames for all of the major polyomavirus proteins (FIG. 1A). The organization is typical for a member of the Polyomaviridae family with an early region consisting of regulatory small-T (ST-Ag) and large-T antigens (LT-Ag) and a late region coding for the VP1, VP2, and VP3 structural proteins. Both the regulatory and structural proteins of MXPyV differ substantially in amino acid sequence from those of other polyomaviruses, with identities ranging from 13-44% (Table 1). Phylogenetic analysis of the VP1, VP2, ST-Ag, and LT-Ag proteins of MXPyV reveals that the taxonomic placement of MXPyV varies from protein to protein (FIG. 2). In VP1 and the large T-antigen, MXPyV shares the most homology with the recently described new human polyomaviruses (HPyV6, HPyV7, WU, and KI), whereas in VP2 or the small T-antigen, MXPyV clusters with the rodent polyomaviruses or forms an independent phylogenetic branch, respectively.

TABLE 1
Amino acid identities of the VP1, VP2, and large T-antigen of MXPyV relative to
that of other polyomaviruses.

| | MX-PyV | Bovine | MurinePneumo | Murine | SV40 | Crow | Finch | WU | Merkel | TSPyV | HPyV6 | HPyV9 |
|---|---|---|---|---|---|---|---|---|---|---|---|---|
| VP1 | | | | | | | | | | | | |
| MX-PyV | | 38 | 38 | 39 | 41 | 44 | 39 | 27 | 36 | 43 | 24 | 44 |
| Bovine | 38 | | 46 | 50 | 50 | 55 | 50 | 26 | 43 | 54 | 25 | 50 |
| MurinePneumo | 38 | 48 | | 44 | 48 | 51 | 48 | 24 | 42 | 50 | 23 | 48 |
| Murine | 39 | 50 | 44 | | 50 | 52 | 51 | 25 | 48 | 55 | 24 | 54 |
| SV40 | 41 | 50 | 48 | 50 | | 54 | 54 | 29 | 46 | 54 | 23 | 53 |
| Crow | 44 | 55 | 51 | 52 | 54 | | 60 | 28 | 46 | 53 | 24 | 59 |
| Finch | 39 | 50 | 48 | 51 | 54 | 60 | | 29 | 46 | 57 | 27 | 56 |
| WU | 27 | 28 | 24 | 25 | 29 | 26 | 29 | | 25 | 24 | 34 | 27 |
| Merkel | 36 | 43 | 42 | 48 | 46 | 46 | 46 | 25 | | 50 | 24 | 63 |
| TSPyV | 43 | 54 | 50 | 55 | 54 | 53 | 57 | 24 | 50 | | 24 | 60 |
| HPyV6 | 24 | 25 | 23 | 24 | 23 | 24 | 27 | 34 | 24 | 24 | | 25 |
| HPyV9 | 44 | 50 | 46 | 54 | 53 | 59 | 56 | 27 | 53 | 60 | 25 | |
| VP2 | | | | | | | | | | | | |
| MX-PyV | | 27 | 27 | 29 | 28 | 30 | 26 | 13 | 23 | 40 | 13 | 33 |
| Bovine | 27 | | 43 | 31 | 28 | 32 | 29 | 13 | 21 | 34 | 17 | 33 |
| MurinePneumo | 27 | 43 | | 26 | 27 | 28 | 26 | 13 | 19 | 30 | 15 | 30 |
| Murine | 29 | 31 | 26 | | 31 | 34 | 30 | 14 | 24 | 42 | 16 | 35 |
| SV40 | 28 | 28 | 27 | 31 | | 33 | 27 | 16 | 22 | 36 | 16 | 31 |
| Crow | 30 | 32 | 28 | 34 | 33 | | 33 | 15 | 22 | 35 | 15 | 35 |
| Finch | 26 | 29 | 26 | 30 | 27 | 33 | | 13 | 20 | 31 | 17 | 29 |
| WU | 13 | 13 | 13 | 14 | 16 | 15 | 13 | | 11 | 12 | 26 | 12 |
| Merkel | 23 | 21 | 19 | 24 | 22 | 22 | 20 | 11 | | 30 | 13 | 25 |
| TSPyV | 40 | 34 | 30 | 42 | 36 | 35 | 31 | 12 | 30 | | 16 | 47 |
| HPyV6 | 13 | 17 | 15 | 16 | 16 | 15 | 17 | 26 | 13 | 16 | | 15 |
| HPyV9 | 33 | 33 | 30 | 35 | 31 | 35 | 29 | 12 | 25 | 47 | 15 | |
| LARGE T | | | | | | | | | | | | |
| ANTIGEN | | | | | | | | | | | | |
| MX-PyV | | 39 | 35 | 34 | 41 | 27 | 29 | 42 | — | 44 | 42 | 43 |
| Bovine | 39 | | 39 | 30 | 39 | 30 | 32 | 42 | — | 36 | 39 | 39 |
| MurinePneumo | 35 | 39 | | 30 | 39 | 26 | 27 | 39 | — | 35 | 33 | 35 |
| Murine | 34 | 30 | 30 | | 32 | 23 | 23 | 33 | — | 40 | 30 | 38 |
| SV40 | 41 | 39 | 39 | 32 | | 25 | 28 | 49 | — | 40 | 37 | 39 |
| Crow | 27 | 30 | 26 | 23 | 25 | | 47 | 29 | — | 26 | 26 | 26 |
| Finch | 29 | 32 | 27 | 23 | 28 | 47 | | 30 | — | 26 | 27 | 28 |
| WU | 42 | 42 | 39 | 33 | 49 | 29 | 30 | | — | 43 | 38 | 40 |
| Merkel | — | — | — | — | — | — | — | — | | — | — | — |
| TSPyV | 44 | 36 | 35 | 40 | 40 | 26 | 28 | 43 | — | | 40 | 48 |
| HPyV6 | 42 | 39 | 33 | 30 | 37 | 26 | 27 | 38 | — | 40 | | 39 |
| HPyV9 | 43 | 39 | 35 | 38 | 39 | 26 | 28 | 40 | — | 48 | 39 | |

### Regulatory Region

Situated between the early and late regions of polyomaviruses is a non-coding regulatory region which contains the origin of replication as well as transcriptional promoters/enhancers. Typical of nearly all polyomaviruses, the regulatory region of MXPyV was found to contain an AT-rich region on the late side of the replication origin (nt 26-57). However, only three T antigen-binding sites, defined by the conserved pentameric GAGGC sequence, were identified in the regulatory region, unlike most polyomaviruses, which contain four to seven such sites. Two of the three T-antigen binding sites in the MXPyV regulatory region were found to combine to form a pentanucleotide palindrome (GAGGCN₄GCCTC) (SEQ ID NO: 38), a feature found in most polyomaviruses. Among the 9 known human polyomaviruses, only HPyV6 (n=2) and HPyV7 (n=1) have fewer T-antigen binding sites than MxPyV.

### Early Region

As typical for polyomaviruses, the LT-Ag of MXPyV is spliced. The donor and acceptor splice site for the LT-Ag of MXPyV were determined based on splice consensus sequences and alignment with the LT-Ag of other polyomaviruses (FIG. 1A). The T-antigen locus of MXPyV contains features conserved with other polyomavirus T antigens, including CR1 (LXXLL), DnaJ (HPDKGG), a pRB 1-binding motif (LXCXE), two PP2A binding sites (CXCX₂C), a zinc finger domain (CX₂CX₅HX₃H), and a helicase/adenosine triphosphatase (ATPse) domain (GPX₃GKT) (FIG. 1B). The nuclear localization signal and host range domain, though present in SV40, BK, and JC virus (12, 24, 33, 40, 52), do not appear to be conserved in MXPyV.

### Late Region

MXPyV retains the core features common to all known polyomaviruses in the late region, including open reading frames for the VP1, VP2, and VP3 capsid proteins, encoding of VP3 in the same ORF as VP2 bp use of an internal start codon, and an overlap between VP1 and VP3. Unlike BKV, JCV, SV40, and SV 12, there is no ORF for an agnoprotein upstream of the VP2 gene.

### Example 2

### Prevalence of MX Polyomavirus in Clinical Samples

A real-time RT-PCR assay targeting the VP1 gene was designed to investigate the prevalence of MX polyomavirus in clinical samples (Tables 2 and 5). The inclusion of the reverse transcriptase step greatly improved the sensitivity of detection of MXPyV (Table 5), presumably by enhancing detection of viral mRNA transcripts in infected host cells. RT-PCR results were confirmed by visualization of an expected-size band on gel electrophoresis, melting curve analysis, and sequencing. All positive results were also independently confirmed using two additional conventional RT-PCR assays targeting a different region of the VP1 and LT-Ag genes. MXPyV was detected in stool samples from children with or without diarrhea on two continents, with prevalence rates of 12.5% (12 of 96) in Mexico, 3.3% (18 of 546) in California, and 4.2% (4 of 96) in Chile. Sequence variation within the 138 nt fragment varied from 0.0-4.3% (data not shown). Analysis of MXPyV-positive stools from Mexico using the ViroChip pan-viral microarray and diarrheal virus PCR identified known pathogenic diarrheal viruses in 50% (6 of 12) samples (Supplementary Table 1).

SUPPLEMENTARY TABLE 1

| MXPyV-Positive | Virochip | | Diarrheal Virus |
|---|---|---|---|
| Sample # | Microarray | PCR | Present? |
| 1 | — | — | − |
| 2 | — | — | − |
| 3 | — | — | − |
| 4 | TTV* | — | − |
| 5 | rotavirus A | rotavirus A | + |
| 6 | norovirus | norovirus | + |
| 7 | rotavirus A | rotavirus A | + |
| 8 | rotavirus A | rotavirus A | + |
| 9 | — | — | − |
| 10 | rotavirus A, | rotavirus A, | + |
| | adenovirus | adenovirus | |
| 11 | astrovirus | astrovirus | + |
| 12 | — | — | − |

*TTV is considered a non-pathogenic virus
SUPPLEMENTARY TABLE 1.
Other diarrheal viruses found in MXPyV-positive samples (12 of 96, 12.5%) from children in Mexico with acute gastroenteritis.
Abbreviations:
TTV, torque teno virus.

Among the MXPyV-positive samples from California for which clinical and demographic data were available, no association was noted between diarrhea and MxPyV infection (Table 3). Interestingly, a child from California was found to be MXPyV-positive both at the time of an acute gastroenteritis episode and 3 months later, suggesting that persistent viral shedding of MXPyV in stool may occur (Table 4). In addition, girls overall were found to be more likely infected by MxPyV than boys (p=0.012) (Table 4). Given the known association of BK and JC virus with disease or asymptomatic shedding in immunocompromised individuals, a screening for MXPyV in 480 plasma and urine samples from transplant patients at a single hospital in California was conducted, with all samples testing negative. Furthermore, 136 respiratory samples from Mexico from hospitalized children with pneumonia were screened, with only one sample (0.74%) confirmed positive for MXPyV infection (Table 2). This sample corresponded to a child with pneumonia who was also found to be co-infected with a rhinovirus/enterovirus by RT-PCR.

Detection of MXPyV, as well as closely related strains MWPyV and HPyV10, appears largely confined to the gastrointestinal tract. MXPyV exhibited an overall prevalence of 3.4% in fecal samples collected from California, Mexico, and Chile (Table 2), although one respiratory sample out of 136 (0.74%) also tested positive. SV40, BKV, JCV, and MCV have also been detected in human feces, although their primary sites of pathology are elsewhere in the human body, as have polyomaviruses WU and KI. These experiments did not detect MXPyV in 480 plasma or urine samples from highly immunocompromised transplant recipients, indicating that these are not reservoir sites for MXPyV infection, as is the case for JC and BK viruses. No association between MXPyV presence and diarrhea was detected in the California and Chile gastroenteritis studies for which controls were available (Tables 2 and 5). In fact, in the samples from Chile, the trend was reversed, with 4 MXPyV-positive samples among 96 asymptomatic control individuals and no positives among 96 children with diarrhea (Table 2). Notably, 6 of 12 MXPyV-positive diarrheal samples from Mexico tested negative by a broad-spectrum viral microarray and specific PCR assays for all known diarrheal viruses, suggesting that MXPyV, if human-tropic, may be a cause of gastroenteritis.

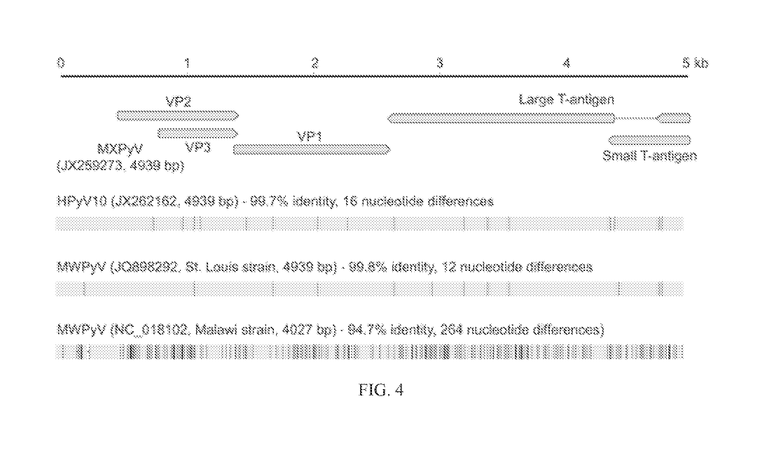

Several lines of evidence indicate that the virus is likely human-tropic. The enhanced sensitivity of RT-PCR over PCR for detection of MXPyV (Table 5) suggests that expressed viral mRNA, presumably present in infected host cells in the feces, is being detected, implying that viral replication occurs in the human gut. In addition, the detection of MXPyV in a child at the time of an acute gastroenteritis episode and 3 months later suggests that, in analogy with other human polyomaviruses, chronic infection by MXPyV is possible. The detection of a closely related variant to MXPyV, HPyV10, in tissue from a patient with WHIM syndrome also indicates that MXPyV, MWPyV, and HPyV10 are likely human-tropic viruses (FIG. 4).

TABLE 2
Results from Screening of Clinical Samples for MXPyV by PCR.

| | | | # of | # of MXPyV- |
|---|---|---|---|---|
| Geographic | Sample | | Samples | Positive Samples |
| Source | type | Subjects | Tested | (%) |
| Mexico | stool | children with | 96 | 12 (12.5%)* |
| | | diarrhea | | |
| Mexico | nasal | children with | 136 | 1 (0.74%) |
| | washes | respiratory | | |
| | | infection | | |
| California | stool | children with or | 546 | 18 (3.3%) |
| (SIFT**) | | without diarrhea | | |
| California | plasma | transplant | 193 | 0 (0.0%) |
| (UCSF) | | recipients | | |
| California | plasma/ | transplant | 287 | 0 (0.0%) |
| (UCSF) | urine | recipients | | |
| Chile | stool | children with | 96 | 0 (0.0%) |
| | | diarrhea | | |
| Chile | stool | age-/sex-matched | 96 | 4 (4.2%) |
| | | controls | | |
| | | (children without | | |
| | | diarrhea) | | |

*includes initial MXPyV-positive sample identified by deep sequencing.
**Stanford Infection and Familial Transmission Study.

TABLE 3
Symptoms corresponding to MXPyV-virus infected stool samples
compared to uninfected samples in the California SIFT study.

| | Symptoms | | |
|---|---|---|---|
| | Diarrhea | Vomiting | Diarrhea or vomiting |
| Stool Samples (n = 546) | N (%) | N (%) | N (%) |
| MXPyV-positive | 10 (56%) | 9 (50%) | 11 (61%) |
| (n = 18) | | | |
| MXPyV-negative | 271 (51%) | 245 (46%) | 317 (60%) |
| (n = 528) | | | |
| P-value | 0.81 | 0.81 | 1.0 |

TABLE 4
Demographics of individuals who provided stool samples from
the California SIFT study according to MXPyV infection status.

| | At least one positive | Negative | |
|---|---|---|---|
| | stool sample | sample(s)** | Total |
| Total | 17* (4%) | 389 (97%) | 406 |
| Male^{‡} | 4 (24%) | 211 (54%) | 215 |
| Mean age, years | 1.76 | 2.21 | 2.19 |
| Median age, years | 1.59 | 1.06 | 1.08 |
| Age range, years | 0.34-60.8 | 0.87-6.01 | |

*One person provided two samples and 16 provided 1. The individual who provided two samples corresponded to a child who was MXPyV-positive both at the time of an acute diarrheal episode and 3 months later.
**146 people provided two samples and 243 provided one.
^{‡}Gender difference significant at p = 0.012 (Fisher's Exact Test).

TABLE 5
Comparison of quantitative RT-PCR vs. PCR assays for
detection of MXPyV and titers of MXPyV in stool.

| | qRT-PCR (Ct) | qPCR (Ct) | qPCR (copies/mL) |
|---|---|---|---|
| Mex-1 | 35.65 | 38.13 | 708 |
| Mex-2 | 30.92 | 36.40 | 1,800 |
| Mex-3 | 33.25 | — | — |
| Mex-4 | 26.3 | 33.46 | 8,791 |
| Mex-5 | 29.98 | 37.19 | 1,176 |
| Mex-6 | 33.69 | — | — |
| Mex-7 | 30.03 | 37.05 | 1,266 |
| Mex-8 | 27. [symbol] | 34.35 | 5,439 |
| Mex-9 | 33.1 | 37.0 | 1,303 |
| Mex-10 | * | * | * |
| CA-1 | 30.86 | 35.01 | 3,810 |
| CA-2 | 27.81 | — | — |
| CA-3 | 31.95 | 36.72 | 1,515 |
| CA-4 | 33.73 | — | — |
| CA-5 | 32.21 | 38.40 | 612 |
| CA-6 | 35.24 | 37.05 | 1,268 |
| CA-7 | 31.75 | 35.14 | 3,552 |
| CA-8 | 30.74 | — | — |
| CA-9 | 30.97 | 39.81 | 286 |
| CA-10 | 30.6 | — | — |
| CA-11 | 26.68 | 32.45 | 15,075 |
| CA-12 | 31.57 | — | — |
| CA-13 | 38.9 | — | — |
| CA-14 | 34.4 | — | — |
| CA-15 | * | * | * |
| CA-16 | * | * | * |
| CA-17 | * | * | * |
| CA-18 | * | * | * |
| CA-19 | * | * | * |
| CA-20 | * | * | * |
| Chile-1 | 33.06 | * | * |
| Chile-2 | * | * | * |
| Chile-3 | * | * | * |
| Chile-4 | * | * | * |

Abbreviations:
Mex, Mexico;
CA, California.
—, not detected by qPCR.
*, not tested by PCR because of sample unavailability or because sample was negative by qRT-PCR but positive by other quantitative RT-PCR assays.

[symbol]

[symbol]

indicates data missing or illegible when filed

All publications, patents, and patent applications cited herein are hereby incorporated by reference in their entirety for all purposes.

SEQ ID NO: 1
gaggcttaggcctccggcccccggcttatatagaaaaaattttagcttattgttttgctacttaacctcaggta
ggtcaacagctattgttggcaagctattgttggcaagtattggtattaatcacccagacaactcagaagtttcc
acctcttggaggcggtccagagttaacctgtgactgttggcgggaagccaataacagcaactttgacatttcat
cacgagccctttaaacgccctctaggcggagacggaagacaattgactcttggcacggacggaagaggaatgcc
gtctgctcaccttagttaacacgagattttctttggaaatactccaaagtacagtaagtatatgggtgctgtaa
tctctgttattcttgatttaatagaattaatatcagttacaggctttgaagcagaaactataatttcaggggag
gcagctgctatagtagaatcacagctttcatctttggcagttatagaaaatgcttcggcagccgaagttttatc
tacttttggattaaatgaaacctcttattctttattagtaaattttcctcaggcctttgaaaatgctgtatata
ctgctcaattaattcaaactatttctggggctagttctctgattgctgctggtatagaaacgcaaccttttcaa
gtatttgatgctggtagtaatatggctttacagcaatggagaccagactattttgatttatttattcctggata
tagacactttgaatattattttaatgttctttctggttggggagaaagtctagtaaatacagtttctagagctt
tttgggaggctttattatcagaaactagaaacactgcaagacttttagcaagcagtgctgtagataatgtttat
aatgttggtgaacagggactccagaatattcaaaatgctctggtgggattaatagagagtgcaagatgggcttt
aagatttccggggaatgtatatcataatttagaaatgtactatacacaattaccaggccttactcctccacaag
taaggtcaattcaaaggcgcttagaagaagcgagaaattatggaccttctgttcaagtggataattctgaaaac
tcagttttttcagaacatttaagtggagactatatatttagaggagaagctccaggaggagcaagacaaagaca
gactcctgattggatgctacaattaattttaggaattcttggagatataactcctttttttaaagaagttattg
aagaagtagaaggagaggaaaatgccgcctaaaagaaagactgtttgtactaaaactgtttgtaccagagcaga
agcccccaacaaaaaagtctgtgaaaaacccacgtgttccagatcttgcagaatgtcatgtaacaaatgtcctt
gtataccgtgtccagttcctactaaagttcctagaattgtttctaagggtgggatagaagttttaaatataatt
ccagggccggacaccacaatgacagttgaggtcattcttcaaccaaggatgggcaatgatgtaaaaacaaacaa
atggtatggctacagtgatccaataacagtaacaaatactcctggtattaatcagctgcccacttatagtacag
ctaaaattaaactgccacccttaaatgaagatatgacctgtgaaaatttgtatatgtgggaggcagttgttctt
aaaactgaattaataggagttactagcttaataactttgcatactccaggtgttgcagatcctgctactgcgcc
agctttaccagtggaaggggtttcatttcatttttttgcagtgggaggacagcctttagatttacaatattgtg
ttcctgcagctgatattgtttacccagatggaacaggcagttttgcaagttcagggggaactcagcaaacctat
gatgcctcatttaaagccattttagatagagatggatactatcctgtggaggcctgggctccagatccagtaaa
aaatgataattctagatactatggaactgttactggaggtactacaacacctcctgtgcttagtacaacaaatg
ctgtttctacagtattactggatgatagaggtgtgggacccctgtgtaagggagatgggctatatgtaacagca
gtagatatttgtggagtgtttcaaatgcctgataatactaggagacacaggggcctagcaagatactttcaggt
acagcttagacaaagagctgttagaaatccttatcctgtaaatagtttgttaaacagtttactgacaaaacaaa
tacctagcattgatggacagccaatggatgggactgataatcaagtacaggatgtaactgtgttccaaggaaca
gaacctcttccaggagaccccactttaactagacatatggatttgcgttgctgcccgggaaccccagtaacaga
tatgccaagtgacgacactccaacaccagttgctccagcagcaagataatttattgtgaattaattccagagtc
ttgttgtgtatcctcgtcagcatctacacatattaaaatatctttcaatggatcttccccagattctatgtttc
ttcttatatcatgatacattccaaagggtacatatttttcaattgtttctttccaatatcttacattatcatga
atttcaggatgaaatgcaattacaggttgccaccaacaaagaagcaataataatgtaacaccactttgaacaat
tctttttctgagcaactcagaatttttttccaaagaatttcttaagaacaacttaggcctgaaatttatagttt
ttatcatcctagcttgcaaagttggaggaataaaatagtcattcatagtaataatacctgggggaaatatttgg
ctctttttattcacatgttttttttcaagatttacttttacacaaccatccaaatgatcccttaaattatctaa
gttggacatacccattcctggagttaaagatgagttagagccctcgttttgaccttttacatcttcaaaaacaa
ccatatattcatcaattgcacaaccaatttcaaaagctaacttatctggaggacagttaacatttagagttttt
cccccaagcaaatcaagtatagcagcagctacagttgttttaccactattaattgggcctttaaataaaacata
tctcctcttaggtacattttcagtcatggcctttattataaataaaataatttcatcaaaatgaggcatcaata
atgataaccaagctactccagccatgtattgacaaatttcaacctcaccacaaatgtcttccattttttcaaac
atatgtttaaaccttaataccaacagatgttctctggtactttctagtattaataatcttctctgagcagtgac
ccagtctgtagcttgctgacaaattgttttttgatttttacaatccttaaaaagttttgaattaatattttgag
tttcatggaatttataatggtgttttaattttttttgttcacattttgagcacccctctacatcttttgcaaag
tctaataacattcccataagtagcaagggatcctcacattgaacttctactgcaaattcacaaatttgctgcca
attcacaactttatctttttcttcctcacagaaatctgttttacttaaaccatgttcctgactctgtttaatta
atttaaaaggagttttacaaagagcatagtaacattcataggcctttaagcaaccttttactaatagaaagctc
actgtacacagatttgaacaataattttttatagcacttactctatgttttccacttaataataaaaataataa
actgtcaccatcaaattcatgcaaactataaaacatagctttaaattttaaaggcactttttcatacaagaatt
gccccttttcccttgtagtatataaaacaaaagaattcagagttttattactaaaaatagcattactaagaaat
tcaaggagtacttcaggaaaatctacaggattaaagtttcttggcctttttggaggagtacaggtagaattgct
gctggattctctgggtctcttcttaggagtgttggtatcatttgcagtctgagaagcactttgagaaggccccg
gttcttcctcatcactgggagcaaaagattcattacaacttaaatcttcatcccatcctctattaaattcttcc
caccattgatcccattcaggagtcccataagtgggatttccctaaaataaaagagaaagaacttacccacaaaa
ttaattctcccagcaagcgaagcagatcaaattcagtattgtgcattatatgcttccaccaaaagaatgaagta
tagccaaattcttgtccaaaccaaagcaaaaagcatttgtagcaaaaacattcaccccaaacaagacattgctt
ttgtttcgctagtttatcatttctgtgctgctttttaagcaagcagcaaacacagccacatttgtgtcttaata
aatcacttgcacacaaaggccaaatgtatataattttttcctcaaagctaggtcctagcacatctcctaaagtt
actacatcgtctataaagtaaccaacctttgcaggaaaataaacttctccttctcttctaagcttttcaattgt
agtatacattttagaaaacggctcattcaagcgtttcattttttctccatctccccctttgtcagggtgcagtt
ttaaacaagtctgcctgtatttatattgcattaagggaatatttccccaagcagcagtatttaagcttaaaaga
gccataagctcttttacttcatctctagaaagtactctatccatccttgctgaatttgcaagtagtaaaaagtt
tgcagacgcggtaaagatggctcccagagtccttcctcttttcaccggaaagaca
SEQ ID NO: 2 (358 . . . 1290 of SEQ ID NO: 1)
atgggtgctgtaatctctgttattcttgatttaatagaattaatatcagttacaggctttgaagcagaaactat
aatttcaggggaggcagctgctatagtagaatcacagctttcatctttggcagttatagaaaatgcttcggcag
ccgaagttttatctacttttggattaaatgaaacctcttattctttattagtaaattttcctcaggcctttgaa
aatgctgtatatactgctcaattaattcaaactatttctggggctagttctctgattgctgctggtatagaaac
gcaaccttttcaagtatttgatgctggtagtaatatggctttacagcaatggagaccagactattttgatttat
ttattcctggatatagacactttgaatattattttaatgttctttctggttggggagaaagtctagtaaataca
gtttctagagctttttgggaggctttattatcagaaactagaaacactgcaagacttttagcaagcagtgctgt
agataatgtttataatgttggtgaacagggactccagaatattcaaaatgctctggtgggattaatagagagtg
caagatgggctttaagatttccggggaatgtatatcataatttagaaatgtactatacacaattaccaggcctt
actcctccacaagtaaggtcaattcaaaggcgcttagaagaagcgagaaattatggaccttctgttcaagtgga
taattctgaaaactcagttttttcagaacatttaagtggagactatatatttagaggagaagctccaggaggag
caagacaaagacagactcctgattggatgctacaattaattttaggaattcttggagatataactccttttttt
aaagaagttattgaagaagtagaaggagaggaaaatgccgcctaa
SEQ ID NO: 3 (688 . . . 1290 of SEQ ID NO: 1)
atggctttacagcaatggagaccagactattttgatttatttattcctggatatagacactttgaatattattt
taatgttctttctggttggggagaaagtctagtaaatacagtttctagagctttttgggaggctttattatcag
aaactagaaacactgcaagacttttagcaagcagtgctgtagataatgtttataatgttggtgaacagggactc
cagaatattcaaaatgctctggtgggattaatagagagtgcaagatgggctttaagatttccggggaatgtata
tcataatttagaaatgtactatacacaattaccaggccttactcctccacaagtaaggtcaattcaaaggcgct
tagaagaagcgagaaattatggaccttctgttcaagtggataattctgaaaactcagttttttcagaacattta
agtggagactatatatttagaggagaagctccaggaggagcaagacaaagacagactcctgattggatgctaca
attaattttaggaattcttggagatataactcctttttttaaagaagttattgaagaagtagaaggagaggaaa
atgccgcctaa
SEQ ID NO: 4 (1280 . . . 2491 of SEQ ID NO: 1)
atgccgcctaaaagaaagactgtttgtactaaaactgtttgtaccagagcagaagcccccaacaaaaaagtctg
tgaaaaacccacgtgttccagatcttgcagaatgtcatgtaacaaatgtccttgtataccgtgtccagttccta
ctaaagttcctagaattgtttctaagggtgggatagaagttttaaatataattccagggccggacaccacaatg
acagttgaggtcattcttcaaccaaggatgggcaatgatgtaaaaacaaacaaatggtatggctacagtgatcc
aataacagtaacaaatactcctggtattaatcagctgcccacttatagtacagctaaaattaaactgccaccct
taaatgaagatatgacctgtgaaaatttgtatatgtgggaggcagttgttcttaaaactgaattaataggagtt
actagcttaataactttgcatactccaggtgttgcagatcctgctactgcgccagctttaccagtggaaggggt
ttcatttcatttttttgcagtgggaggacagcctttagatttacaatattgtgttcctgcagctgatattgttt
acccagatggaacaggcagttttgcaagttcagggggaactcagcaaacctatgatgcctcatttaaagccatt
ttagatagagatggatactatcctgtggaggcctgggctccagatccagtaaaaaatgataattctagatacta
tggaactgttactggaggtactacaacacctcctgtgcttagtacaacaaatgctgtttctacagtattactgg
atgatagaggtgtgggacccctgtgtaagggagatgggctatatgtaacagcagtagatatttgtggagtgttt
caaatgcctgataatactaggagacacaggggcctagcaagatactttcaggtacagcttagacaaagagctgt
tagaaatccttatcctgtaaatagtttgttaaacagtttactgacaaaacaaatacctagcattgatggacagc
caatggatgggactgataatcaagtacaggatgtaactgtgttccaaggaacagaacctcttccaggagacccc
actttaactagacatatggatttgcgttgctgcccgggaaccccagtaacagatatgccaagtgacgacactcc
aacaccagttgctccagcagcaagataa
SEQ ID NO: 5 = reverse complement of (2493 . . . 4259) and (4615 . . . 4854) of SEQ ID NO: 1
ggaaatcccacttatgggactcctgaatgggatcaatggtgggaagaatttaatagagga
tgggatgaagatttaagttgtaatgaatcttttgctcccagtgatgaggaagaaccgggg
ccttctcaaagtgcttctcagactgcaaatgataccaacactcctaagaagagacccaga
gaatccagcagcaattctacctgtactcctccaaaaaggccaagaaactttaatcctgta
gattttcctgaagtactccttgaatttcttagtaatgctatttttagtaataaaactctg
aattcttttgttttatatactacaagggaaaaggggcaattcttgtatgaaaaagtgcct
ttaaaatttaaagctatgttttatagtttgcatgaatttgatggtgacagtttattattt
ttattattaagtggaaaacatagagtaagtgctataaaaaattattgttcaaatctgtgt
acagtgagctttctattagtaaaaggttgcttaaaggcctatgaatgttactatgctctt
tgtaaaactccttttaaattaattaaacagagtcaggaacatggtttaagtaaaacagat
ttctgtgaggaagaaaaagataaagttgtgaattggcagcaaatttgtgaatttgcagta
gaagttcaatgtgaggatcccttgctacttatgggaatgttattagactttgcaaaagat
gtagaggggtgctcaaaatgtgaacaaaaaaaattaaaacaccattataaattccatgaa
actcaaaatattaattcaaaactttttaaggattgtaaaaatcaaaaaacaatttgtcag
caagctacagactgggtcactgctcagagaagattattaatactagaaagtaccagagaa
catctgttggtattaaggtttaaacatatgtttgaaaaaatggaagacatttgtggtgag
gttgaaatttgtcaatacatggctggagtagcttggttatcattattgatgcctcatttt
gatgaaattattttatttataataaaggccatgactgaaaatgtacctaagaggagatat
gttttatttaaaggcccaattaatagtggtaaaacaactgtagctgctgctatacttgat
ttgcttgggggaaaaactctaaatgttaactgtcctccagataagttagcttttgaaatt
ggttgtgcaattgatgaatatatggttgtttttgaagatgtaaaaggtcaaaacgagggc
tctaactcatctttaactccaggaatgggtatgtccaacttagataatttaagggatcat
ttggatggttgtgtaaaagtaaatcttgaaaaaaaacatgtgaataaaaagagccaaata
tttcccccaggtattattactatgaatgactattttattcctccaactttgcaagctagg
atgataaaaactataaatttcaggcctaagttgttcttaagaaattctttggaaaaaaat
tctgagttgctcagaaaaagaattgttcaaagtggtgttacattattattgcttctttgt
tggtggcaacctgtaattgcatttcatcctgaaattcatgataatgtaagatattggaaa
gaaacaattgaaaaatatgtaccctttggaatgtatcatgatataagaagaaacatagaa
tctggggaagatccattgaaagatattttaatatgtgtagatgctgacgaggatacacaa
caagactctggaattaattcacaataaatggatagagtactttctagagatgaagtaaaa
gagcttatggctcttttaagcttaaatactgctgcttggggaaatattcccttaatgcaa
tataaatacaggcagacttgtttaaaactgcaccctgacaaagggggagatggagaaaaa
atgaaacgcttgaatgagccgttttctaaaatgtatactacaattgaaaagcttagaaga
gaaggagaagtttattttcctgcaaag
SEQ ID NO: 6 = reverse complement of (4234 . . . 4854) of SEQ ID NO: 1
atggatagagtactttctagagatgaagtaaaagagcttatggctcttttaagcttaaat
actgctgcttggggaaatattcccttaatgcaatataaatacaggcagacttgtttaaaa
ctgcaccctgacaaagggggagatggagaaaaaatgaaacgcttgaatgagccgttttct
aaaatgtatactacaattgaaaagcttagaagagaaggagaagtttattttcctgcaaag
gttggttactttatagacgatgtagtaactttaggagatgtgctaggacctagctttgag
gaaaaaattatatacatttggcctttgtgtgcaagtgatttattaagacacaaatgtggc
tgtgtttgctgcttgcttaaaaagcagcacagaaatgataaactagcgaaacaaaagcaa
tgtcttgtttggggtgaatgtttttgctacaaatgctttttgctttggtttggacaagaa
tttggctatacttcattcttttggtggaagcatataatgcacaatactgaatttgatctg
cttcgcttgctgggagaattaattttgtgggtaagttctttctcttttattttagggaaa
tcccacttatgggactcctga
SEQ ID NO: 7 = reverse complement of (4615 . . . 4854) of SEQ ID NO: 1
atggatagagtactttctagagatgaagtaaaagagcttatggctcttttaagcttaaat
actgctgcttggggaaatattcccttaatgcaatataaatacaggcagacttgtttaaaa
ctgcaccctgacaaagggggagatggagaaaaaatgaaacgcttgaatgagccgttttct
aaaatgtatactacaattgaaaagcttagaagagaaggagaagtttattttcctgcaaag

### REFERENCES

1. Abed, Y., D. Wang, and G. Boivin. 2007. WU polyomavirus in children, Canada. Emerg Infect Dis 13:1939-1941.
2. Agelli, M., L. X. Clegg, J. C. Becker, and D. E. Rollison. 2010. The etiology 427 and epidemiology of merkel cell carcinoma. Curr Probl Cancer 34:14-37.
3. Albores-Saavedra, J., K. Batich, F. Chable-Montero, N. Sagy, A. M. Schwartz, and D. E. Henson. 2010. Merkel cell carcinoma demographics, morphology, and survival based on 3870 cases: a population based study. J Cutan Pathol 37:20-27.
4. Allander, T., K. Andreasson, S. Gupta, A. Bjerkner, G. Bogdanovic, M. A. Persson, T. Dalianis, T. Ramqvist, and B. Andersson. 2007. Identification of a third human polyomavirus. J Virol 81:4130-4136.
5. Babakir-Mina, M., M. Ciccozzi, C. Alteri, P. Polchi, A. Picardi, F. Greco, G. Lucarelli, W. Arcese, C. F. Perno, and M. Ciotti. 2009. Excretion of the novel polyomaviruses KI and WU in the stool of patients with hematological disorders. Med Virol 81:1668-1673.
6. Babakir-Mina, M., M. Ciccozzi, C. F. Perno, and M. Ciotti. 2011. The novel KI, WU, MC polyomaviruses: possible human pathogens? New Microbiol 34:1-8.
7. Barron-Romero, B. L., J. Barreda-Gonzalez, R. Doval-Ugalde, J. Zermeno-Eguia Liz, and M. Huerta-Pena. 1985. Asymptomatic rotavirus infections in day care centers. J Clin Microbiol 22:116-118.
8. Bialasiewicz, S., D. M. Whitey, S. B. Lambert, K. Jacob, C. Bletchly, D. Wang, M. D. Nissen, and T. P. Sloots. 2008. Presence of the newly discovered human polyomaviruses KI and WU in Australian patients with acute respiratory tract infection. J Clin Virol 41:63-68.
9. Braun, W. E. 2003. BK polyomavirus: a newly recognized threat to transplanted kidneys. Cleve Clin J Med 70:1056, 1059-1060, 1062 passim.
10. Chen, E. C., S. Yagi, K. R. Kelly, S. P. Mendoza, R. P. Tarara, D. R. Canfield, N. Maninger, A. Rosenthal, A. Spinner, K. L. Bales, D. P. Schnurr, N. W. Lerche, and C. Y. Chiu. 2011. Cross-species transmission of a novel adenovirus associated with a fulminant pneumonia outbreak in a new world monkey colony. PLoS Pathog 7:e1002155.
11. Chen, T., L. Hedman, P. S. Mattila, T. Jartti, O. Ruuskanen, M. Soderlund-Venermo, and K. Hedman. 2011. Serological evidence of Merkel cell polyomavirus primary infections in childhood. J Clin Virol 50:125-129.
12. Chen, Y., P. M. Sharp, M. Fowkes, O. Kocher, J. T. Joseph, and I. J. Koralnik. 2004. Analysis of 15 novel full-length BK virus sequences from three individuals: evidence of a high intra-strain genetic diversity. J Gen Virol 85:2651-2663.
13. Chiu, C. Y., A. L. Greninger, E. C. Chen, T. D. Haggerty, J. Parsonnet, E. Delwart, J. L. Derisi, and D. Ganem. 2010. Cultivation and serological characterization of a human Theiler's-like cardiovirus associated with diarrheal disease. J Virol 84:4407-4414.
14. Chiu, C. Y., A. L. Greninger, K. Kanada, T. Kwok, K. F. Fischer, C. Runckel, J. K. Louie, C. A. Glaser, S. Yagi, D. P. Schnurr, T. D. Haggerty, J. Parsonnet, D. Ganem, and J. L. DeRisi. 2008. Identification of cardioviruses related to Theiler's murine encephalomyelitis virus in human infections. Proc Natl Acad Sci USA 105:14124-14129.
15. Chiu, C. Y., S. Rouskin, A. Koshy, A. Urisman, K. Fischer, S. Yagi, D. Schnurr, P. B. Eckburg, L. S. Tompkins, B. G. Blackburn, J. D. Merker, B. K. Patterson, D. Ganem, and J. L. DeRisi. 2006. Microarray detection of human parainfluenzavirus 4 infection associated with respiratory failure in an immunocompetent adult. Clin Infect Dis 43:e71-76.
16. Crandall, K. A., M. Perez-Losada, R. G. Christensen, D. A. McClellan, and R. P. Viscidi. 2006. Phylogenomics and molecular evolution of polyomaviruses. Adv Exp Med Biol 577:46-59.
17. DeCaprio, J. A., J. W. Ludlow, J. Figge, J. Y. Shew, C. M. Huang, W. H. Lee, E. Marsilio, E. Paucha, and D. M. Livingston. 1988. SV40 large tumor antigen forms a specific complex with the product of the retinoblastoma susceptibility gene. Cell 54:275-283.
18. Delwart, E. L. 2007. Viral metagenomics. Rev Med Virol 17:115-131.
19. Drummond, A., B. Ashton, M. Cheung, J. Heled, M. Kearse, R. Moir, S. Stones-Havas, S. Sturrock, T. Thierer, and A. Wilson. 2010. Geneious v5.5.6, Available from http://www.geneious.com.
20. Drummond, A. J., and A. Rambaut. 2007. BEAST: Bayesian evolutionary analysis by sampling trees. BMC Evol Biol 7:214.
21. Eash, S., K. Manley, M. Gasparovic, W. Querbes, and W. J. Atwood. 2006. The human polyomaviruses. Cell Mol Life Sci 63:865-876.
22. Eisen, M. B., P. T. Spellman, P. O. Brown, and D. Botstein. 1998. Cluster analysis and display of genome-wide expression patterns. Proc Natl Acad Sci USA 95:14863-14868.
23. Feng, H., M. Shuda, Y. Chang, and P. S. Moore. 2008. Clonal integration of a polyomavirus in human Merkel cell carcinoma. Science 319:1096-1100.
24. Frisque, R. J. 2001. Structure and function of JC virus T′ proteins. J Neurovirol 7:293-297.
25. Gardner, S. D., A. M. Field, D. V. Coleman, and B. Hulme. 1971. New human papovavirus (B.K.) isolated from urine after renal transplantation. Lancet 1:1253-1257.
26. Gaynor, A. M., M. D. Nissen, D. M. Whitey, I. M. Mackay, S. B. Lambert, G. Wu, D. C. Brennan, G. A. Storch, T. P. Sloots, and D. Wang. 2007. Identification of a novel polyomavirus from patients with acute respiratory tract infections. PLoS Pathog 3:e64.
27. Gjoerup, 0., and Y. Chang. 2010. Update on human polyomaviruses and cancer. Adv Cancer Res 106:1-51.
28. Greninger, A. L., E. C. Chen, T. Sittler, A. Scheinerman, N. Roubinian, G. Yu, E. Kim, D. R. Pillai, C. Guyard, T. Mazzulli, P. Is a, C. F. Arias, J. Hackett, G. Schochetman, S. Miller, P. Tang, and C. Y. Chiu. 2010. A metagenomic analysis of pandemic influenza A (2009 H₁N₁) infection in patients from North America. PLoS One 5:e13381.
29. Greninger, A. L., L. Holtz, G. Kang, D. Ganem, D. Wang, and J. L. DeRisi. 2010. Serological evidence of human klassevirus infection. Clin Vaccine Immunol 17:1584-1588.
30. Han, T. H., J. Y. Chung, J. W. Koo, S. W. Kim, and E. S. Hwang. 2007. WU polyomavirus in children with acute lower respiratory tract infections, South Korea. Emerg Infect Dis 13:1766-1768.
31. Hou, J., and E. O. Major. 2000. Progressive multifocal leukoencephalopathy: JC virus induced demyelination in the immune compromised host. J Neurovirol 6 Suppl 2:S98-S100.
32. Houben, R., M. Shuda, R. Weinkam, D. Schrama, H. Feng, Y. Chang, P. S. Moore, and J. C. Becker. 2010. Merkel cell polyomavirus-infected Merkel cell carcinoma cells require expression of viral T antigens. J Virol 84:7064-7072.
33. Ishii, N., N. Minami, E. Y. Chen, A. L. Medina, M. M. Chico, and H. Kasamatsu. 1996. Analysis of a nuclear localization signal of simian virus 40 major capsid protein Vpl. J Virol 70:1317-1322.
34. Katoh, K., K. Kuma, H. Toh, and T. Miyata. 2005. MAFFT version 5: improvement in accuracy of multiple sequence alignment. Nucleic Acids Res 33:511-518.
35. Kazem, S., E. van der Meijden, S. Kooijman, A. S. Rosenberg, L. C. Hughey, J. C. Browning, G. Sadler, K. Busam, E. Pope, T. Benoit, P. Fleckman, E. de Vries, J. A. Eekhof, and M. C. Feltkamp. 2012. Trichodysplasia spinulosa is characterized by active polyomavirus infection. J Clin Virol 53:225-230.
36. Kean, J. M., S. Rao, M. Wang, and R. L. Garcea. 2009. Seroepidemiology of human polyomaviruses. PLoS Pathog 5:e1000363.
37. Knowles, W. A. 2006. Discovery and epidemiology of the human polyomaviruses BK virus (BKV) and JC virus (JCV). Adv Exp Med Biol 577:19-45.
38. Loyo, M., R. Guerrero-Preston, M. Brait, M. O. Hogue, A. Chuang, M. S. Kim, R Sharma, N. J. Liegeois, W. M. Koch, J. A. Califano, W. H. Westra, and D. Sidransky. 2010. Quantitative detection of Merkel cell virus in human tissues and possible mode of transmission. Int J Cancer 126:2991-2996.
39. Mendez-Toss, M., D. D. Griffin, J. Calva, J. F. Contreras, F. I. Puerto, F. Mota, H. Guiscafre, R. Cedillo, O. Munoz, I. Herrera, S. Lopez, and C. F. Arias. 2004. Prevalence and genetic diversity of human astroviruses in Mexican children with symptomatic and asymptomatic infections. J Clin Microbiol 42:151-157.
40. Moreland, R. B., H. G. Nam, L. M. Hereford, and H. M. Fried. 1985. Identification of a nuclear localization signal of a yeast ribosomal protein. Proc Natl Acad Sci USA 82:6561-6565.
41. Padgett, B. L., D. L. Walker, G. M. ZuRhein, R. J. Eckroade, and B. H. Dessel. 1971. Cultivation of papova-like virus from human brain with progressive multifocal leucoencephalopathy. Lancet 1:1257-1260.
42. Pallas, D. C., L. K. Shahrik, B. L. Martin, S. Jaspers, T. B. Miller, D. L. Brautigan, and T. M. Roberts. 1990. Polyoma small and middle T antigens and SV40 small t antigen form stable complexes with protein phosphatase 2A. Cell 60:167-176.
43. Pipas, J. M. 1992. Common and unique features of T antigens encoded by the polyomavirus group. J Virol 66:3979-3985.
44. Randhawa, P., A. Vats, and R. Shapiro. 2006. The pathobiology of polyomavirus infection in man. Adv Exp Med Biol 577:148-159.
45. Ren, L., R. Gonzalez, Z. Xie, J. Zhang, C. Liu, J. Li, Y. Li, Z. Wang, X. Kong, Y. Yao, Y. Hu, S. Qian, R. Geng, Y. Yang, G. Vernet, G. Paranhos-Baccala, Q. Jin, K. Shen, and J. Wang. 2008. WU and KI polyomavirus present in the respiratory tract of children, but not in immunocompetent adults. J Clin Virol 43:330-333.
46. Ren, L., R. Gonzalez, X. Xu, J. Li, J. Zhang, G. Vernet, G. Paranhos-Baccala, Q. Jin, and J. Wang. 2009. WU polyomavirus in fecal specimens of children with acute gastroenteritis, China. Emerg Infect Dis 15:134-135.
47. Reploeg, M. D., G. A. Storch, and D. B. Clifford. 2001. Bk virus: a clinical review. Clin Infect Dis 33:191-202.
48. Ronquist, F., M. Teslenko, P. van der Mark, D. L. Ayres, A. Darling, S. Hohna, B. Larget, L. Liu, M. A. Suchard, and J. P. Huelsenbeck. 2012. MrBayes 3.2: efficient Bayesian phylogenetic inference and model choice across a large model space. Syst Biol 61:539-542.
49. Schowalter, R. M., D. V. Pastrana, K. A. Pumphrey, A. L. Moyer, and C. B. Buck. 2010. Merkel cell polyomavirus and two previously unknown polyomaviruses are chronically shed from human skin. Cell Host Microbe 7:509-515.
50. Scuda, N., J. Hofmann, S. Calvignac-Spencer, K. Ruprecht, P. Liman, J. Kuhn, H. Hengel, and B. Ehlers. 2011. A novel human polyomavirus closely related to the african green monkey-derived lymphotropic polyomavirus. J Virol 85:4586-4590.
51. Shuda, M., H. Feng, H. J. Kwun, S. T. Rosen, O. Gjoerup, P. S. Moore, and Y. Chang. 2008. T antigen mutations are a human tumor-specific signature for Merkel cell polyomavirus. Proc Natl Acad Sci USA 105:16272-16277.
52. Spence, S. L., and J. M. Pipas. 1994. Simian virus 40 large T antigen host range domain functions in virion assembly. J Virol 68:4227-4240.
53. Tang, P., and C. Chiu. 2010. Metagenomics for the discovery of novel human viruses. Future Microbiol 5:177-189.
54. van der Meijden, E., R. W. Janssens, C. Lauber, J. N. Bouwes Bavinck, A. E. Gorbalenya, and M. C. Feltkamp. 2010. Discovery of a new human polyomavirus associated with trichodysplasia spinulosa in an immunocompromized patient. PLoS Pathog 6:e1001024.
55. Vanchiere, J. A., S. Abudayyeh, C. M. Copeland, L. B. Lu, D. Y. Graham, and J. S. Butel. 2009. Polyomavirus shedding in the stool of healthy adults. J Clin Microbiol 47:2388-2391.
56. Vanchiere, J. A., Z. S. White, and J. S. Butel. 2005. Detection of BK virus and simian virus 40 in the urine of healthy children. J Med Virol 75:447-454.
57. Walter, G., R. Ruediger, C. Slaughter, and M. Mumby. 1990. Association of protein phosphatase 2A with polyoma virus medium tumor antigen. Proc Natl Acad Sci USA 87:2521-2525.
58. Wattier, R. L., M. Vazquez, C. Weibel, E. D. Shapiro, D. Ferguson, M. L. Landry, and J. S. Kahn. 2008. Role of human polyomaviruses in respiratory tract disease in young children. Emerg Infect Dis 14:1766-1768.
59. Yuan, X. H., Y. Jin, Z. P. Xie, H. C. Gao, Z. Q. Xu, L. S. Zheng, R. F. Zhang, J. R. Song, Y. D. Hou, and Z. J. Duan. 2008. Prevalence of human KI and WU polyomaviruses in children with acute respiratory tract infection in China. J Clin Microbiol 46:3522-3525.

1. A cDNA molecule comprising a nucleotide sequence of at least 100 nucleotides in length, wherein said sequence has at least 90% sequence identity to a portion of the same length in SEQ ID NO:1 or its complement.

2. The cDNA molecule of claim 1, wherein said sequence has at least 95% identity to a portion of the same length in SEQ ID NO:1 or its complement.

3. The cDNA molecule of claim 1, wherein said sequence has 100% identity to a portion of the same length in SEQ ID NO:1 or its complement.

4. The cDNA molecule of claim 1, wherein the nucleotide sequence comprises at least 90% identity over the full length of SEQ ID NO:1 or its complement.

5. The cDNA molecule of claim 1, wherein the nucleotide sequence comprises at least 95% identity over the full length of SEQ ID NO:1 or its complement.

6. The cDNA molecule of claim 1, wherein the nucleotide sequence comprises SEQ ID NO:1.

7. An isolated expression vector comprising the cDNA molecule of claim 1.

8. An isolated host cell comprising the expression vector of claim 6.

9. An cDNA molecule comprising a nucleotide sequence of at least 100 nucleotides in length, said sequence having at least 90% sequence identity to an open reading frame selected from the group consisting of SEQ ID NOs:2-7.

10. The cDNA molecule of claim 9, wherein the nucleotide sequence has at least 95% identity to the open reading frame selected from the group consisting of SEQ ID NOs:2-7.

11. The cDNA molecule of claim 9, wherein the nucleotide sequence has an open reading frame selected from the group consisting of SEQ ID NOs:2-7.

12. A recombinant peptide encoded by the cDNA molecule of claim 9.

13. An isolated antibody that specifically binds to the recombinant peptide of claim 12.

14. The antibody of claim 13, wherein the antibody is a polyclonal antibody.

15. The antibody of claim 13, wherein the antibody is a monoclonal antibody.

16. A method for detecting a polyomavirus in a biological sample, the method comprising the steps of:
(a) contacting the biological sample with a recombinant primer that hybridizes to a portion of a nucleotide sequence selected from SEQ ID NO:1-7 and complement of SEQ ID NO:1;
(b) performing a nucleic acid amplification reaction to generate an amplicon; and
(c) detecting the amplicon with a cDNA probe that hybridizes under stringent hybridization conditions with a nucleic acid sequence selected from SEQ ID NO: 1-7 and complement of SEQ ID NO:1.

17. A method for detecting a MXPyV antigen in a biological sample, the method comprising the steps of:
(a) contacting the biological sample with a recombinant immobilized antibody specific for the MXPyV antigen for a time and under conditions sufficient to allow the formation of a complex of the recombinant antibody with its target antigen; and
(b) detecting the binding of said recombinant antibody to MXPyV antigen in the biological sample,
wherein said recombinant antibody is immobilized on a solid phase and the presence of said binding is indicative of a MXPyV antigen in the biological sample.

18. The method of claim 17, wherein step (b) comprises: adding a conjugate comprising a recombinant antibody specific for an MXPyV antigen attached to a signal generating compound capable of detecting a detectable signal, wherein said antibody binds to a different epitope of MXPyV than the recombinant immobilized antibody; and
detecting the presence of MXPyV antigen in the sample by detecting the signal generated by the signal-generating compound.

19. A method of detecting an anti-MXPyV antibody in a human biological sample comprising the steps of:
(a) contacting the sample suspected of containing an anti-MXPyV antibody with an immobilized recombinant peptide of claim 12 for a time and under conditions sufficient to allow the formation of a complex of the anti-MXPyV antibody with said recombinant peptide;
(b) adding a conjugate for a time and under conditions sufficient to allow the conjugate to bind to the anti-MXPyV antibody of the complex, the conjugate comprising an anti-human antibody attached to a signal generating compound capable of generating a detectable signal;
(c) detecting the presence of the anti-MXPyV antibody in the sample by detecting the signal generated by the signal generating compound.

20. A method of detecting an anti-MXPyV antibody in a human biological sample comprising the steps of:
(a) contacting the sample suspected of containing an anti-MXPyV antibody with a with an immobilized recombinant anti-human antibody, under time and conditions sufficient to allow the formation of a complex of the anti-MXPyV antibody with said immobilized recombinant antibody;
(b) adding a conjugate for a time and under conditions sufficient to allow the conjugate to bind to the anti-MXPyV antibody of the complex, the conjugate comprising a peptide of claim 12 being attached to a signal generating compound capable of generating a detectable signal; and
(c) detecting the presence of the anti-MXPyV antibody in the sample by detecting the signal generated by the signal generating compound.

21. A method of detecting an anti-MXPyV antibody in a biological sample comprising the steps of:
(a) contacting the sample suspected of containing the anti-MXPyV antibody with an immobilized recombinant anti-human antibody, under time and conditions sufficient to allow the formation of a complex of the anti-MXPyV antibody with said immobilized antibody;
(b) adding a recombinant peptide of claim 12 for a time and under conditions sufficient to allow the peptide to bind to the anti-MXPyV antibody of the complex;
(c) adding a conjugate for a time and under conditions sufficient to allow the conjugate to bind to the recombinant peptide bound to the anti-MXPyV of the complex, the conjugate comprising a recombinant anti-MXPyV antibody attached to a signal generating compound capable of detecting a detectable signal; and
(d) detecting the presence of the anti-MXPyV in the sample by detecting the signal generated by the signal-generating compound.

22. An immunogenic composition comprising the isolated recombinant peptide of claim 12.

23. A kit comprising at least one synthetic primer that hybridizes to a nucleotide sequence comprising SEQ ID NO:1.

24. A kit comprising the isolated recombinant peptide of claim 12.

25. A kit comprising the recombinant antibody of claim 13.
